(19) United States
(12) Patent Application Publication
(10) Pub. No.: US 2007/0225318 A1
(43) Pub. Date: Sep. 27, 2007

(54) Pyrazole Compounds Useful In The Treatment Of Inflammation

(75) Inventors: Peter Nilsson, Solna (SE); Andrei Sanin, Solna (SE); Benjamin Pelcman, Solna (SE)

Correspondence Address: MORGAN LEWIS & BOCKIUS LLP, 1111 PENNSYLVANIA AVENUE NW, WASHINGTON, DC, 20004, US

(73) Assignee: BIOLIPOX AB, Solna (SE)

(21) Appl. No.: 11/663,208

(22) PCT Filed: Sep. 19, 2005
(86) PCT No.: PCT/GB05/03580

§ 371 Date: Mar. 19, 2007

Related U.S. Application Data
(60) Provisional application No. 60/610,952, filed on Sep. 20, 2004.
(60) Provisional application No. 60/617,081, filed on Oct. 12, 2004.

(51) Int. Cl. A61K 31/4155; A61K 31/4375; A61K 31/47; C07D 215/00; C07D 231/10; C07D 471/04

(52) U.S. Cl. 514/300; 514/314; 514/406; 546/122; 546/152; 548/364.1

(57) ABSTRACT

There is provided compounds of formula (I), wherein R¹, R², R^{a} and R^{b} have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

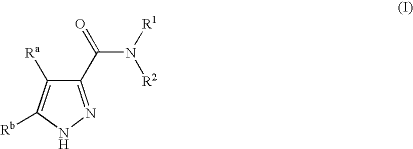

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory, pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

International patent application WO 2004/080999 discloses numerous 1-substituted pyrazole derivatives for use in the treatment of inflammation. One of the compounds used in the synthesis of certain compounds that are exemplified in that document is 1H-pyrazole-3-carboxylic acid benzo[1,3]dioxol-5-ylamide. The use of the latter compound as a pharmaceutical is neither mentioned nor suggested.

Pyrazole compounds are disclosed in U.S. Pat. No. 5,919,776 for use as modulators of chemokine receptor activity and in international patent application WO 97/30034 as anticancer agents. The use of the compounds in the treatment of inflammation is neither mentioned nor suggested in either of these documents.

Certain pyrazole compounds that are structurally related to those described herein are commercially available. However, to the knowledge of the applicant, these compounds have never been disclosed in any printed publication and as such have no perceived utility ascribed to them.

International patent application WO 98/41518 this discloses various compounds, including N-(2-methyl-1,2,3,4-tetrahydroisoquinolin-7-yl)-4-trifluoromethyl-pyrazole-3-carboxamide, for use as anticonvulsants. This document does not mention or suggest the use of these compounds in the treatment of inflammation.

International patent application WO 01/57024 discloses various pyrazoles that are useful in blockading voltage-dependent sodium channels, and international patent applications WO 01/58869 and WO 03/020217 disclose various nitrogen-containing heterocycles, including pyrazoles, that are useful as cannabinoid receptors. There is no mention in any of these documents of the use of such compounds in treating inflammation and/or as inhibitors of lipoxygenases.

International patent application WO 2005/016877 discloses pyrazoles that may be useful in the inhibition of 11β-hydroxysteroid dehydrogenase-1 (and therefore useful in the treatment of inter alia diabetes). There is no specific disclosure in this document of pyrazoles that are substituted in the 3-position with an aromatic amido group.

Certain pyrazolecarboxylic acid hydrazides, structurally unrelated to the compounds described herein, have been disclosed as anti-inflammatory agents in Tihanyi et al, Eur. J. Med. Chem.-Chim. Ther., 1984, 19, 433 and Goel et al, J. Chem. Inf. Comput. Sci. 1995, 35, 510.

International patent application WO 96/11917 discloses various compounds that may be useful in treating inflammation. This document only discloses benzoxazoles and benzothiazoles substituted, directly or via an alkyl linker group, in the 2-position by an aromatic group.

International application WO 03/037274 discloses various pyrazoles that may be useful in treating inflammatory pain, which mechanism works by blocking sodium channels. This document only specifically discloses pyrazoles that are N-substituted and/or pyrazoles that are substituted by an amido group in the 4-position.

International application WO 03/068767 also relates to inter alia pyrazole-containing compounds that may be useful in treating inflammatory pain by opening potassium ion channels. However, this document only discloses pyrimidinyl amido compounds.

Vertuani et al., Journal of Pharmaceutical Sciences, Vol. 74, No. 9 (1985) discloses various pyrazoles that possess anti-inflammatory and analgesic activities. There is no mention or suggestion of pyrazoles that are substituted in the 3-position by a heteroaromatic amido group.

### DISCLOSURE OF THE INVENTION

According to the invention there is provided a compound of formula I, 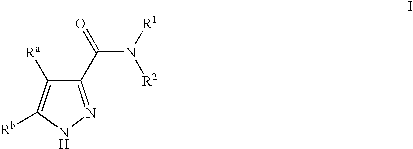
wherein
R¹ represents a bicyclic heterocyclic group, which group is optionally substituted by one or more substituents selected from B¹ and comprises:
(a) 1 to 4 heteroatoms selected from nitrogen, oxygen and sulfur;
(b) a 5- or 6-membered aromatic ring, which ring is attached to the rest of the compound of formula I via the essential —N(R²)— group of the latter; and
(c) a 5- or 6-membered aromatic, or a 4- to 8-membered non-aromatic, ring, which ring is attached to the other ring via two atoms that are common to both rings and are adjacent to each other;
   R² represents H or C₁₋₆ alkyl, which latter group is optionally substituted by one or more halo groups;
   B¹ represents —OH, cyano, halo, nitro, C₁₋₆ alkyl, (which latter group is optionally substituted by one or more halo atoms), —OR^{3x}, —N(R^{3a})R^{3b}, —C(O)R^{3c}, —C(O)OR^{3d}, —C(O)N(R^{3e})R^{3f}, —N(R^{3g})C(O)R^{3h}, —N(R³ⁱ)C(O)N(R^{3j})R^{3k}, —N(R^{3m})S(O)₂R^{4a}, —S(O)ₚR^{4b}, —OS(O)₂R^{4c}, —S(O)₂N(R³ⁿ)R^{3p} or, provided that it is not attached to a ring that is aromatic in its nature, ═O;
   R^{a} and R^{b} independently represent H, halo, cyano, C₁₋₆ alkyl (which alkyl group is optionally substituted by one or more halo or C₁₋₆ alkoxy groups (which alkoxy group may itself be substituted by one or more halo group)), C₁₋₆ alkoxy (which alkoxy group is optionally substituted by one or more halo atoms) or —N(R^{3q})R^{3r};
   R^{3a} to R^{3r} and R^{4b} independently represent H or C₁₋₆ alkyl (which alkyl group is optionally substituted by one or more halo atoms);
   R^{4a}, R^{4c} and R^{3x} independently represent C₁₋₆ alkyl (which alkyl group is optionally substituted by one or more halo atoms); or
   any pair of R^{3a}, R^{3b}, R^{3e} to R^{3r} and R^{4a} may, for example when present on the same or adjacent atoms, be linked together to form, with those, or other relevant, atoms, a 3- to 8-membered (e.g. 5- to 6-membered) ring, optionally containing a further 1 to 3 (e.g. 1) heteroatoms and/or 1 to 3 unsaturations, which ring is itself optionally substituted by one or more substituents selected from halo and C₁₋₆ alkyl (which alkyl group is optionally substituted by one or more halo atoms); or
   when R¹ is substituted by two —OR^{3x} groups that are adjacent to each other, these groups may be linked to form, together with the oxygen atoms to which they are attached, a 5- or 6-membered ring optionally containing 1 further heteroatom (e.g. nitrogen) and 1 unsaturation, which ring is itself optionally substituted by one or more substituents selected from halo and C₁₋₆ alkyl (which alkyl group is optionally substituted by one or more halo atoms); and
   p represents 0, 1 or 2;
   or a pharmaceutically-acceptable salt thereof,
   provided that, when R^{b} and R² both represent H, and:
(A) R^{a} represents H, then R¹ does not represent a benzo[1,3]dioxol-5-yl group;
(B) R^{a} represents chloro, then R¹ does not represent a benzothiazol-2-yl group;
(C) R^{a} represents iodo, then R¹ does not represent a benzothiazol-2-yl, a 6-methoxybenzothiazol-2-yl, or a 1-methyl-1H-benzoimidazol-2-yl, group;
(D) R^{a} represents bromo, then R¹ does not represent a 5,6-dihydro-4H-cyclopenta[b]thiophene-3-carboxylic acid ethyl ester group;
(E) R^{a} represents trifluoromethyl, then R¹ does not represent a 2-methyl-1,2,3,4-tetrahydroisoquinolin-7-yl group;
(F) R^{a} represents iodo, then R¹ does not represent a 2-mercapto-4-oxoquinazolin-3yl group,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The term “unsaturations” when used herein refers to triple or, preferably double bonds.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups and C_{1-q} alkoxy (including —OR^{3x} groups) groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group or a C_{2-q}-cycloalkoxy group). Further, when there is a sufficient number (i.e. a minimum of three or four as appropriate) of carbon atoms, such alkyl and alkoxy groups may also be part cyclic/acyclic. Such allyl and alkoxy groups may also be saturated or, when there is a sufficient number (i.e. a minimum of two) of carbon atoms, be unsaturated (forming, for example in the case of the alkyl group, a C_{2-q} alkenyl or a C_{2-q} alkynyl group).

For the avoidance of doubt, alkoxy groups are attached to the rest of the molecule via the essential oxygen atom of that group.

The term “halo”, when used herein, includes fluoro, chloro, bromo and iodo.

For the avoidance of doubt, in cases in which the identity of two or more substituents in a compound of the invention may be the same, the actual identities of the respective substituents are not in any way interdependent. For example, in the situation in which two or more B¹ substituents on an R¹ group represent C₁₋₆ alkyl, the alkyl groups in question may be the same or different, i.e. the identities of the two B¹ groups are not to be regarded as being interdependent.

R¹ groups that may be mentioned include those in which in which the total number of atoms in the ring system is between nine and fourteen (e.g. between ten and twelve). In this respect, the total number of atoms in the R¹ bicycle may be nine, ten or eleven. Further, the total number of heteroatoms in the R¹ group (i.e. not including the possible substituents on this group) is preferably between 1 and 3.

Any non-aromatic rings in R¹ groups may be saturated or unsaturated, containing one or more double and/or triple bonds.

5- to 6-membered aromatic rings that may be mentioned include phenyl, furanyl, imidazolyl, isothiazolyl, isoxazolyl, oxadiazolyl (including 1,2,3-oxadiazolyl, 1,2,4-oxadiazolyl and 1,3,4-oxadiazolyl), oxazolyl, pyrazinyl, pyrazolyl, pyridazinyl, pyridyl, pyrimidinyl, pyrrolyl, tetrazolyl, thiadiazolyl (including 1,2,3-thiadiazolyl, 1,2,4-thiadiazolyl and 1,3,4-thiadiazolyl), thiazolyl, thienyl, triazolyl (including 1,2,3-triazolyl, 1,2,4-triazolyl and 1,3,4-triazolyl) and the like.

4- to 8-membered non-aromatic rings may be bridged. That is, any two non-adjacent atoms of the ring may be linked by either an alkylene or heteroalkylene chain. The term “alkylene” in this context refers to (—CH₂—)ₙ groups in which n may represent 1, 2, 3, etc, as appropriate, and the term “heteroalkylene” refers to an alkylene group in which at least one of the carbon atoms has been replaced with a heteroatom (such as oxygen, sulfur or nitrogen).

The skilled person will appreciate that when such heteroalkylene chains contain a nitrogen atom, then that atom may be further substituted in order for the rules of valency to be adhered to. Accordingly, such nitrogen atoms may be further attached to a hydrogen atom (i.e. unsubstituted), or substituted by an appropriate B¹ substituent as hereinbefore defined (e.g. optionally substituted C₁₋₆ alkyl as defined herein). Alternatively, such nitrogen atoms may be unsubstituted and linked via one double and one single bond (so forming a —N═ linkage).

4- to 8-membered non-aromatic rings that may be mentioned include thiopyranyl, tetrahydrothiopyranyl (including S,S-dioxotetrahydrothiopyranyl), norbornanyl, 7-azabicyclo[2.2.1]heptanyl, 6-azabicyclo[3.1.1]heptanyl, 6-azabicyclo[3.2.1]-octanyl, 8-azabicyclo[3.2.1]octanyl, 7-oxabicyclo[2.2.1]heptanyl, 6-oxabicyclo[3.2.1]octanyl, quinuclidinyl, tropanyl or, more preferably, azetidinyl, cyclobutyl, cyclopentyl, cyclopentenyl, cyclopentadienyl, cyclohexyl, cyclohexenyl, cyclohexadienyl, cycloheptyl, cycloheptenyl, cycloheptadienyl, cyclooctyl, cyclooctenyl, cyclooctadienyl, cyclooctatrienyl, cyclooctynyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, oxetanyl, oxiranyl, piperazinyl, piperidinyl, pyranyl, pyrazolidinyl, pyrrolidinonyl, pyrrolidinyl, pyrrolinyl, sulfolanyl, 3-sulfolenyl, tetrahydropyranyl, tetrahydrofuranyl, tetrahydropyridyl, thietanyl, thiiranyl, thiolanyl, thiomorpholinyl, trithianyl (including 1,3,5-trithianyl) and the like.

Compounds of the invention that may be mentioned include those in which:

R^{4b} represents C₁₋₆ alkyl (which alkyl group is optionally substituted by one or more halo atoms);

when R¹ is substituted with two —OR^{3x} groups that are adjacent to each other, then the appropriate pair of R^{3x} groups are not linked as hereinbefore defined.

Preferred compounds of the invention include those in which the 5- or 6-membered aromatic ring of R¹ that is attached to —N(R²)— contains less than 3 (e.g. less than 2) heteroatoms and, most preferably 1 or no heteroatoms. In the instances where this ring contains a heteroatom, the heteroatom is preferably selected from nitrogen and sulfur. Thus, preferred rings include phenyl, pyridyl (e.g. 3-pyridyl, 4-pyridyl or, more preferably, 2-pyridyl) and thienyl (e.g. 2-thienyl) rings.

Preferred compounds of the invention include those in which the 5- or 6-membered aromatic or 4- to 8-membered non-aromatic ring, which is attached to the other, essential aromatic ring contains less than 3 heteroatoms. When such rings are 5-membered aromatic rings, they preferably contain 1 or 2 heteroatoms. When such rings are non-aromatic rings, they are preferably 5- to 7-membered. When these non-aromatic groups are 5-membered, they preferably contain 0 or 2 heteroatoms, and when these groups are 7-membered, they preferably contain no heteroatoms. Thus, preferred rings include isothiazolyl, cyclopentyl, tetrahydrothiopyranyl (e.g. tetrahydrothiopyranyl), norbornanyl, piperidinyl, tetrahydropyranyl, pyrazinyl, imidazolyl, cycloheptyl or, more particularly, pyridyl, phenyl, pyrrolyl, pyridazinyl, cyclohexyl, thiazolyl, pyrazolyl, dioxolanyl (e.g. 1,3-dioxolanyl) and dioxanyl (e.g. 1,4-dioxanyl) rings.

R¹ groups that may be mentioned thus include cyclopentapyridyl, dihydrocyclopentapyridyl (including 6,7-dihydro-5H-cyclopenta[b]pyridyl), thiopyranopyridyl, tetrahydrothiopyranopyridyl (including 5,6,7,8-tetrahydro-5H-thiopyrano[4,3-b]pyridyl (e.g. 6,6-dioxo-5,6,7,8-tetrahydro-6λ⁶-thiopyrano[4,3-b]pyrid-3-yl)), azatricycloundecatrienyl (including 3-azatricyclo-[6.2.1.0^{2,7}]undeca-2(7),3,5-trienyl), tetrahydronaphthyridinyl (including 5,6,7,8-tetrahydro[1,6]naphthyridinyl), pyranopyridyl, dihydropyranopyridyl (including 7,8-dihydro-5H-pyrano[4,3-b]pyridyl), cycloheptapyridyl, tetrahydrocyclo-heptapyridyl (including 6,7,8,9-tetrahydro-5H-cyclohepta[b]pyridyl), thienopyridyl, dihydrothienopyridyl (including 4,7-dihydro-5H-thieno[2,3-c]pyridyl), tetrahydrocycloheptathienyl (including 5,6,7,8-tetrahydro-4H-cyclohepta[b]-thienyl) or, more preferably, benzimidazolyl, benzodioxanyl, benzodioxepinyl, benzodioxolyl (including 1,3-benzodioxolyl), benzofuranyl, benzofurazanyl, benzomorpholinyl, benzopyranyl, benzothiazolyl, benzothiadiazolyl (including 2,1,3-benzothiadiazolyl), benzothienyl, benzoxadiazolyl (including 2,1,3-benzoxadiazolyl), benzoxazinyl (including 3,4-dihydro-2H-1,4-benzoxazinyl), benzoxazolyl, chromanyl, cinnolinyl, dihydrocyclopentathienyl (including 5,6-dihydro-4H-cyclopenta[b]thienyl), imidazo[1,2-a]pyridyl, indazolyl, indolinyl, indolyl, isobenzofuranyl, isochromanyl, isoindolinyl, isoindolyl, isoquinolinyl, isothiochromanyl, naphthyridinyl (including 1,6-naphthyridinyl or, more particularly, 1,5-naphthyridinyl and 1,8-naphthyridinyl), phthalazinyl, pteridinyl, purinyl, quinazolinyl, quinolinyl, quinolizinyl, quinoxalinyl, tetrahydrobenzothienyl (including 4,5,6,7-tetrahydrobenzothienyl), tetrahydrocycloheptathienyl (including 5,6,7,8-tetrahydro-4H-cyclohepta[b]thienyl), tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), thiochromanyl and the like.

The point of attachment of R¹ groups may be via any atom of the essential aromatic ring including (where appropriate) a heteroatom (such as a nitrogen atom) in that ring. R¹ groups may also be in the N- or S-oxidised form.

Thus, preferred values of R¹ include benzothiadiazolyl (including 2,1,3-benzothiadiazol-4-yl), tetrahydroquinolinyl (including 5,6,7,8-tetrahydroquinolin-3-yl), dihydrocyclopentapyridyl (including 6,7-dihydro-5H-cyclopenta[b]pyrid-3-yl), tetrahydrothiopyranopyridyl (including 6,6-dioxo-5,6,7,8-tetrahydro-6λ⁶-thiopyrano[4,3-b]pyrid-3-yl), azatricycloundecatrienyl (including 3-azatricyclo-[6.2.1.0^{2,7}]undeca-2(7),3,5-trien-5-yl), tetrahydronapthyridinyl (including 5,6,7,8-tetrahydro[1,6]naphthyridin-3-yl), dihydropyranopyridyl (including 7,8-dihydro-5H-pyrano[4,3-b]pyrid-3-yl), quinoxalinyl (including quinoxalin-5-yl), benzimidazolyl (including benzimidazol-5-yl), tetrahydrocycloheptapyridyl (including 6,7,8,9-tetrahydro-5H-cyclohepta[b]pyrid-3-yl), dihydrothienopyridyl (including 2-[4,7-dihydro-5H-thieno[2,3-c]pyridyl]), tetrahydrocycloheptathienyl (including 5,6,7,8-tetrahydro-4H-cyclohepta[b]thien-2-yl), naphthyridinyl (including 1,6-naphthyridin-3-yl) or, more preferably, quinolinyl (including quinolin-4-yl or, more particularly, quinolin-2-yl, quinolin-3-yl, quinolin-5-yl, quinolin-6-yl and quinolin-8-yl), isoquinolinyl (including isoquinolin-1-yl, isoquinolin-4-yl, isoquinolin-8-yl or, more particularly, isoquinolin-3-yl and isoquinolin-5-yl), benzodioxolyl (including benzo[1,3]dioxol-4-yl and benzo[1,3]dioxol-5-yl), indolyl (including indol-4-yl and indol-5-yl), cinnolinyl (including cinnolin-5-yl), benzodioxanyl (including 2,3-dihydrobenzo[1,4]dioxan-6-yl), tetrahydrobenzothienyl (including 4,5,6,7-tetrahydrobenzo[b]thien-2-yl), benzothiazolyl (including benzothiazol-5-yl or, more particularly, benzothiazol-6-yl) and indazolyl (including indazol-5-yl and, more particularly, indazol-6-yl).

Substituents on R¹ groups may, where appropriate, be located on any atom in the ring system including a heteroatom.

Preferred substituents on R¹ include C₁₋₃ alkoxy (e.g. methoxy), ═O, —OH, —SH, —C(O)R^{3c}, in which R^{3c} represents C₁₋₃ alkyl (such as methyl), two adjacent OR^{3x} groups that are linked together to form a 5- or 6-membered ring (e.g. a 1,3-dioxolyl or 1,4-dioxanyl ring), or, the substituents on R¹ are, more preferably, halo (such as chloro, bromo or, more particularly, fluoro), C₁₋₄ (e.g. C₁₋₃) alkyl (such as tert-butyl or, more particularly, methyl) optionally substituted by one or more halo (e.g. fluoro) atoms (so forming, for example, a trifluoromethyl group), C(O)OR^{3d}, in which R^{3d} represents C₁₋₄ (e.g. C₁₋₃) alkyl (such as tert-butyl or, more particularly, ethyl), and cyano.

Preferred compounds of the invention include those in which:

when any pair of R^{3a}, R^{3b}, R^{3e} to R^{3r} and R^{4a} are linked to form a ring, then the ring so formed is an unsubstituted, saturated 5- or 6-membered ring, optionally containing one further heteroatom (e.g. oxygen). For example, R^{3a} and R^{3b} and/or R^{3q} and R^{3r} may independently be linked to form, together with the essential nitrogen atom to which each respective pair is attached, a morpholinyl, piperidinyl or pyrrolidinyl group;

when two R^{3x} groups are linked, they form a 5- or 6-membered unsubstituted ring containing no further heteroatoms and/or no unsaturations (so forming, for example, a dioxoly or a dioxanyl group);

when p represents 1 or 2, then R^{4b} represents C₁₋₆ alkyl (which alkyl group is optionally substituted by one or more halo atoms).

Further preferred compounds of the invention include those in which:

R² represents H;

R^{a} and R^{b} independently represent halo (e.g. bromo, chloro or fluoro) or, more preferably, H or C₁₋₃ alkyl (such as methyl), which latter group is substituted by one or more halo (e.g. fluoro) groups (so forming, for example, a trifluoromethyl group) or, is preferably unsubstituted.

For example:

R^{a} may represent H, methyl, bromo or fluoro;

R^{b} may represent H, methyl, trifluoromethyl or chloro.

Substituents on the 5- or 6-membered aromatic ring of R¹ that is attached to —N(R²)— are preferably selected from methyl, cyano, —C(O)O-ethyl and —OH (or oxo; e.g. when the compound may tautomerise). Substituents on the 5- or 6-membered aromatic or 4- to 8-membered non-aromatic ring of R¹, which is attached to the other, essential aromatic ring are preferably selected from fluoro, chloro, bromo, methyl, tert-butyl, trifluoromethyl, ═O, —SH, —OH, —OCH₃, —C(O)O-tert-butyl, —C(O)CH₃, 1,3-dioxolyl and 1,4-dioxanyl.

Particularly preferred compounds of the invention include those of the examples described hereinafter.

Compounds of formula I may be made in accordance with techniques that are well known to those skilled in the art, for example as described hereinafter.

According to a further aspect of the invention there is provided a process for the preparation of a compound of formula I as follows:

(i) For compounds of formula I in which R^{b} represents C₁₋₆ alkyl, optionally substituted as hereinbefore defined, or halo, reaction of a corresponding compound of formula I in which R^{b} represents hydrogen, with an appropriate base (or a mixtures of bases), such as potassium bis(trimethylsilyl)amide, sodium bis(trimethylsilyl)amide, sodium hydride, potassium tert-butoxide or an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)) followed by quenching with an appropriate electrophile such as:
(a) for compounds of formula I in which R^{b} represents an optionally substituted C₁₋₆ alkyl group, a compound of formula II,
   R^{c}L^{1a} II
wherein R^{c} represents C₁₋₆ alkyl (which alkyl group is optionally substituted by one or more halo or C₁₋₆ alkoxy groups (which alkoxy group may itself be substituted by one or more halo group)), and L^{1a} represents a suitable leaving group such as halo (e.g. iodo or bromo) or a sulfonate group (such as —OSO₂CF₃, OSO₂CH₃ and —OSO₂-aryl (e.g. —O-tosyl)); or
(b) for compounds of formula I in which R^{b} represents halo, an electrophile that provides a source of halide ions. For example, for bromide ions, reagents include N-bromosuccinimide, bromine and 1,2-dibromotetrachloroethane, for chloride ions reagents include N-chlorosuccinimide, chlorine, iodine monochloride and hexachloroethane, for iodide ions, appropriate reagents include iodine, diiodoethane and diiodotetrachloroethane and for fluoride ions reagents include xenon difluoride, SELECTFLUOR® ([1-(chloromethyl)-4-fluoro-1,4-diazonia-bicyclo[2.2.2]octane bis(tetrafluoroborate)]), CF₃OF, and perchloryl fluoride.

The skilled person will appreciate that the corresponding compounds of formula I in which R^{b} represents hydrogen (on which the above reaction is performed) may need to be protected at the nitrogen atom of the pyrazole ring system, preferably with a protective group that is also a directing metallation group (such as a benzenesulfonyl group). The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. when a benzenesulfonyl group is employed, by hydrolysis).

(ii) Reaction of a compound of formula III, 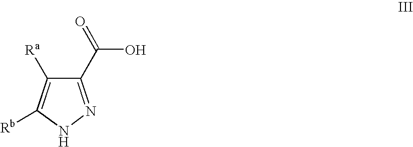
or a N-protected and/or O-protected (e.g. ester) derivative thereof, wherein R^{a} and R^{b} are as hereinbefore defined, with a compound of formula IV,
HN(R¹)(R²) IV
wherein R¹ and R² are as hereinbefore defined under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine 1,8-diazabicyclo [5.4.0]undec-7-ene, sodium hydroxide, N-ethyldiisopropylamine, N-(methylpolystyrene)-4-(methylamino)pyridine, lithium diisopropylamide, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, trifluoromethylbenzene, dioxane or triethylamine) and a suitable coupling agent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)-phosphonium hexafluorophosphate, 2-(1H-benzotriazol-1-yl)-1,1,3,3-tetramethyluronium hexafluorophosphate, benzotriazol-1-yloxytris-pyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate), 1-cyclohexylcarbodiimide-3-propyloxymethyl polystyrene), O-(7-azabenzotriazol-1-yl)-N,N′,N′-tetramethyluronium hexafluorophosphate or O-benzotriazol-1-yl-N,N,N′,N′-tetramethyluronium tetrafluoroborate). Alternatively, compounds of formula III may first be activated by treatment with a suitable reagent (e.g. oxalyl chloride, thionyl chloride, etc) optionally in the presence of an appropriate solvent (e.g. dichloromethane, dimethylformamide, THF, toluene or benzene) and a suitable catalyst (e.g. DMF), resulting in the formation of the respective acyl chloride. This activated intermediate may then be reacted with a compound of formula IV under standard conditions, such as those described above. Alternatively, an azodicarboxylate may be employed under Mitsunobo conditions known to those skilled in the art. The skilled person will appreciate that when compounds of formula IV are liquid in nature, they may serve as both solvent and reactant in this reaction. Alternative methods of performing this step include reaction of an O-protected derivative (e.g. an ethyl ester) of a compound of formula III with a compound of formula IV, which latter compound may first be treated with trimethylaluminum, for example in an inert atmosphere and in the presence of a suitable solvent (e.g. dichloromethane).

(iii) Reaction of a compound of formula V, 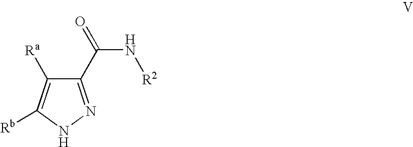
or a N-protected (e.g. at the pyrazole nitrogen) derivative thereof, wherein R^{a}, R^{b} and R² are as hereinbefore defined, with a compound of formula VI,
R¹-L² VI
wherein L² represents a suitable leaving group, such as halo (e.g. chloro, bromo and iodo), —OSO₂CF₃, —B(OH)₂, —Sn(R^{z})₃ (wherein R^{z} is C₁₋₆ alkyl and preferably, methyl or butyl) or —Bi(R¹)₂, and R¹ is as hereinbefore defined, for example in the presence of a catalyst containing, preferably, Pd or Cu, and a base and, optionally, in the presence of solvent and a ligand. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.

(iv) For compounds of formula I in which R^{b} represents hydrogen and R^{a} is as hereinbefore defined, reaction of a compound of formula VII, 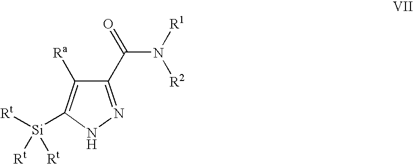
wherein each R^{t} independently represents a C₁₋₆ alkyl (e.g. a methyl or isopropyl) group or an aryl (e.g. phenyl) group, and R^{a}, R¹ and R² are as hereinbefore defined, with an appropriate reagent for the removal of the silyl group, such as a source of halide anions (e.g. tetrabutylammonium fluoride, tetramethylammonium fluoride, hydrogen fluoride or potassium fluoride), for example, in the presence of a suitable solvent (e.g. tetrahydrofuran) at room temperature.

(v) Reaction of a compound of formula VIII, 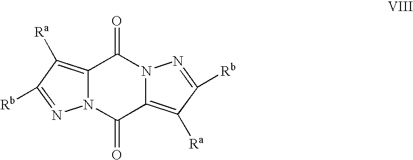
wherein R^{a} and R^{b} are as hereinbefore defined, with a compound of formula IV as hereinbefore defined, for example under coupling conditions such as those described hereinbefore in respect of process step (ii) above. Preferred conditions include reaction in the presence of base, solvent but no coupling reagent. In this case, the compound of formula IV may also be employed in excess.

(vi) For compounds of formula I in which one of R^{a} or R^{b} represents an optionally substituted C₁₋₆ alkyl group and the other represents H, reaction of a corresponding compound of formula I in which one of R^{a} or R^{b} represents bromo or iodo and the other represents H (as appropriate) with a suitable organolithium base (e.g. t-BuLi, s-BuLi or n-BuLi) optionally in the presence of an additive (such as one hereinbefore described in respect of process step (i)), followed by quenching with a compound of formula II, as hereinbefore defined. This reaction may be performed in the presence of a suitable solvent, such as one hereinbefore described in respect of process step (i) at low temperatures (e.g. −78 to −120° C.) under an inert atmosphere.

(vii) For compounds of formula I in which R^{a} and/or R^{b} represent C₁₋₆ alkoxy (optionally substituted as hereinbefore defined), reaction of a compound corresponding to a compound of formula I but in which in place of the relevant substituents, R^{a} and/or R^{b} (as appropriate), (a) hydroxy group(s) is/are present, with a compound of formula II as hereinbefore defined, in which R^{c} represents C₁₋₆ alkyl (optionally substituted by one or more halo substituents), or (for the introduction of a methoxy group at R^{a} and/or R^{b}) with diazomethane. Each reaction may be performed under standard conditions known to those skilled in the art, for example the former may be performed in the presence of base (e.g. sodium hydride) and a suitable solvent (e.g. dimethylformamide or tetrahydrofuran) and the latter may be performed in the presence of a suitable solvent (e.g. an aromatic hydrocarbon such as benzene or a di(alkyl)ether such as diethyl ether). In the case of the latter, diazomethane may be prepared from Diazald® or in situ from trimethylsilyldiazomethane.

(viii) For compounds of formula I in which R^{a} and/or R^{b} represents —N(R^{3q})R^{3r} in which one of R^{3q} or R^{3r} represents H and the other represents methyl or C₂₋₆ alkyl (which latter group is optionally substituted by one or more halo atoms), reaction of a corresponding compound of formula I in which R^{a} and/or R^{b} represents —NH₂, with a compound of formula IX,
R^{3s}C(O)H IX
wherein R^{3s} represents either H or C₁₋₅ alkyl (which alkyl group is optionally substituted by one or more halo atoms) so forming an optionally substituted C₁₋₆ alkyl group (i.e. R^{3q} or R^{3r}, as appropriate). This reaction may be performed under reaction conditions known to those skilled in the art. For example the above amine of formula I and the aldehyde of formula IX may be condensed to form the imine (e.g. under dehydration reaction conditions such as Dean-Stark conditions or reaction in the presence of a dehydrating agent (e.g. magnesium sulfate)) followed by reduction of the imine intermediate (e.g. reaction in the presence of a reducing agent such as sodium borohydride or reaction employing hydrogenation reaction conditions) or the two steps (i.e. the reductive amination) may be performed in “one-pot” (e.g. employing a suitable chemoselective reducing agent such as sodium triacetoxyborohydride or sodium cyanoborohydride). The reaction may be performed in the presence of a suitable solvent such as an alcohol (e.g. methanol or ethanol). For compounds of formula I in which R^{a} and/or R^{b} represents —N(R^{3q})R^{3r} and R^{3q} and R^{3r} each represent an optionally substituted C₁₋₆ alkyl group, the second alkyl group on the nitrogen atom may be introduced using conditions known to those skilled in the art, such as those described below in respect of process step (ix).

(ix) For compounds of formula I in which R^{a} and/or R^{b} represent —N(R^{3q})R^{3r} in which at least one of R^{3q} and R^{3r} represents C₁₋₆ alkyl (optionally substituted as hereinbefore defined) and the other represents H or C₁₋₆ alkyl (optionally substituted as hereinbefore defined), reaction of a corresponding compound of formula I in which R^{a} and/or R^{b} represent(s) —NH₂ (as appropriate) with a compound of formula II in which R^{c} represents C₁₋₆ alkyl optionally substituted by one or more halo substituents, under reaction conditions known to those skilled in the art, such as those described hereinbefore in respect of process step (vii). The skilled person will appreciate that for mono-alkylation the amino group may need to be first protected, in which case the protecting group will need to be removed subsequent to alkylation. For dialkylation where the two alkyl groups are different, the skilled person will appreciate that the amino group may first have to be mono-alkylated.

(x) For compounds of formula I in which R^{a} and/or R^{b} represent —N(R^{3q})R^{3r} in which R^{3q} and R^{3r} are linked together to form a 3- to 8-membered ring, reaction of a corresponding compound of formula I in which R^{a} and/or R^{b} represents —NH₂ (as appropriate) with a compound of formula IXA,
L^{1a}-Z-L^{1a} IXA
wherein Z represents a C₂₋₇ alkylene chain (which alkylene chain optionally contains 1 to 3 heteroatoms and/or 1 to 3 unsaturations, and is optionally substituted by one or more groups selected from halo and C₁₋₆ alkyl (which alkyl group is optionally substituted by one or more halo atoms)), and L^{1a} is as hereinbefore defined (but wherein the values of each respective L^{1a} group are not to be regarded as being interdependent), for example under reaction conditions such as those described hereinbefore in respect of process step (vii).

(xi) For compounds of formula I in which R^{a} and/or R^{b} represents cyano or 1-alkynyl, reaction of a corresponding compound of formula I in which R^{a} and/or R^{b} (as appropriate) represents halo (preferably iodo or bromo) with a compound which is a source of cyano anions (e.g. sodium, potassium, copper (I) or zinc cyamide) for the introduction of the cyano group, or with a 1-alkyne for the introduction of the 1-alkynyl group. The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine).

(xii) For compounds of formula I in which R² represents H, reaction of a compound of formula IXB, 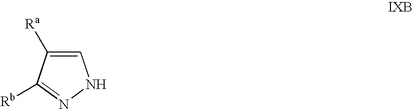
or a N-protected derivative thereof, wherein R^{a} and R^{b} are as hereinbefore defined, with a suitable base, such as one described in process step (i) above, followed by reaction with a compound of formula IXC,
R¹—N═C═O IXC
wherein R¹ is as hereinbefore defined, followed by quenching with a suitable proton source (e.g. water or aqueous, saturated NH₄Cl solution). This reaction may be performed under similar conditions to those described above in respect of process step (i). The skilled person will appreciate that the pyrazole nitrogen may need to be protected. The skilled person will further appreciate that the amido group will be introduced a to one of the pyrazole nitrogen atoms, and thus when R^{b} represents H, there are two alternative positions.

Compounds of formula I in which R^{a} and/or R^{b} represent amino groups, as well as compounds corresponding to compounds of formula I but in which R^{a} and/or R^{b} represent hydroxy groups, may be prepared by analogous reactions to those described hereinafter for the introduction of amino or hydroxy substituents into R^{a} and/or R^{b} positions of compounds of formula III.

Compounds of formula III (or derivatives thereof) in which R^{b} represents H, C₁₋₆ alkyl or C₁₋₆ alkoxy (which latter two groups are optionally substituted as hereinbefore defined) may be prepared by reaction of a compound of formula X, 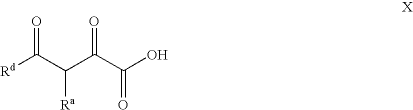
or an enol ether equivalent, or an O-protected derivative thereof, wherein R^{d} represents H, C₁₋₆ alkyl (which alkyl group is optionally substituted by one or more halo or C₁₋₆ alkoxy groups (which alkoxy group may itself be substituted by one or more halo group)) or C₁₋₆ alkoxy (which alkoxy group is optionally substituted by one or more halo atoms) and R^{a} is as hereinbefore defined, with hydrazine (or a hydrate or derivative thereof), for example in the presence of an alcoholic solvent (e.g. ethanol) at elevated temperature (e.g. at reflux).

Compounds of formulae III, V or VIII in which either one of R^{a} or R^{b} represents halo and the other represents H, C₁₋₆ alkyl, C₁₋₆ alkoxy (which latter two groups are optionally substituted as hereinbefore defined) or —N(R^{3q})R^{3r} (wherein R^{3q} and R^{3r} are as hereinbefore defined) or both R^{a} and R^{b} represent halo, may be prepared by reaction of a corresponding compound of formula III, V or VIII (as appropriate) in which R^{a} and R^{b} both represent H or one of R^{a} or R^{b} represents H and the other represents optionally substituted C₁₋₆ alkyl or C₁₋₆ alkoxy or —N(R^{3q})R^{3r} (as appropriate), with an electrophile that provides a source of halide ions, such as one described hereinbefore in respect of process step (i)(b), under reaction conditions known to those skilled in the art. Thus 4-halo, 5-halo or 4,5-dihalo substituted pyrazoles may be prepared in such a manner.

Compounds of formula III in which one of R^{a} or R^{b} represents fluoro and the other represents H may be prepared from 4-nitropyrazole-3-carboxylic acid or 5-nitropyrazole-3-carboxylic acid (as appropriate) employing an appropriate reagent for the conversion of the nitro group to a fluoro group (such as sodium fluoride, potassium fluoride, tetramethylammonium fluoride or tetrabutylammonium fluoride) under conditions known to those skilled in the art.

Compounds of formula III in which one of R^{a} or R^{b} represents amino and the other represents H may be prepared from 4-nitropyrazole-3-carboxylic acid or 5-nitropyrazole-3-carboxylic acid (as appropriate) by conversion of the nitro group to an amino group (employing any suitable reducing conditions such as hydrogenation).

Compounds of formula III in which one of R^{a} or R^{b} represents halo or cyano and the other represents H or compounds corresponding to a compound of formula III but in which one of R^{a} or R^{b} represents hydroxy and the other represents H, may be prepared by reaction of a compound of formula III in which one of R^{a} or R^{b} represents amino and the other represents H (as appropriate) followed by conversion of the amino group to a diazonium salt (employing reagents and conditions known to those skilled in the art, e.g. NaNO₂ and HCl at 5° C.) and then the addition of an appropriate nucleophile for the conversion to a halo, cyano or hydroxy group. Suitable nucleophiles include potassium, sodium or copper halides (for the introduction of the halo group), potassium, sodium or copper cyanides (for the introduction of the cyano group) or water (for the introduction of the hydroxy group).

Compounds corresponding to a compound of formula III but in which R^{b} represents hydroxyl or C₁₋₆ alkoxy (optionally substituted as hereinbefore defined) and R^{a} represents H may be prepared by reaction of a compound of formula XI, 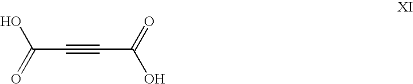
or an O-protected (e.g. an optionally substituted C₁₋₆ alkyl ester) derivative thereof, with hydrazine (or a hydrate or derivative thereof) under reaction conditions known to those skilled in the art.

Compounds of formula III in which R^{a} and R^{b} independently represent H, halo, cyano, C₁₋₆ alkoxy (optionally substituted as hereinbefore defined) or —N(R^{3q})R^{3r} (wherein R^{3q} and R^{3r} are as hereinbefore defined) may alternatively be prepared by oxidation of a compound of formula XII, 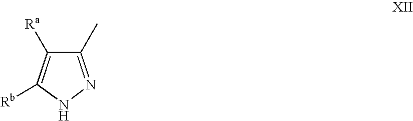
wherein R^{b} and R^{a} independently represent H, halo, cyano, optionally substituted C₁₋₆ alkoxy or —N(R^{3q})R^{3r}, under oxidation conditions known to those skilled in the art, for example mild or strong (e.g. employing an aqueous solution of potassium permanganate and heating at reflux) oxidation conditions as appropriate.

Compounds of formula III in which R^{b} represents halo may be prepared by reaction of a compound of formula XIII, 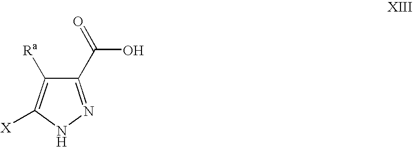
or a N-protected and/or O-protected (e.g. ester) derivative thereof, wherein X represents Si(R^{t})₃ or Sn(R^{z})₃ and R^{a}, R^{t} and R^{z} are as hereinbefore defined, using a suitable halogenating reagent such as cesium fluoride, cesium fluoroxysulfate or one described hereinbefore in respect of process step (i)(b), optionally in the presence of a suitable solvent (e.g. hexane, diethyl ether, tetrahydrofuran or 1,4-dioxane or mixtures thereof) under conditions known to those skilled in the art.

Compounds of formula IV in which R² represents H may be prepared:

(I) by reaction of a compound of formula VIII, as hereinbefore defined, with ammonia, or preferably with a protected derivative thereof (e.g. benzylamine), under conditions such as those described hereinbefore in respect of preparation of compounds of formula I (process step (iii));

(II) by reduction of a compound of formula XIIIA,
R¹—NO₂ XIIIA,
wherein R¹ is as hereinbefore defined, under standard reaction conditions, for example, reduction by hydrogenation in the presence of a catalyst (e.g. palladium on carbon), with a source of hydrogen (e.g. hydrogen gas or nascent hydrogen (e.g. from ammonium formate)), optionally in the presence of a solvent (such as an alcoholic solvent (e.g. methanol)); or
(III) for compounds of formula IV in which R¹ further represents a 6,6- or 5,6-heteroaromatic bicycle in which both rings are themselves aromatic in nature, by aromatisation of a corresponding compound of formula IV in which the 6- or 5-membered ring that is attached to the requisite amino group of the compound of formula IV is aromatic and the other essential ring of R¹ that is attached to this aromatic ring is non-aromatic. Such aromatisation reaction conditions include heating in a solvent (e.g. mesitylene or diglyme), in the presence of a suitable catalyst (e.g. Pd/C), optionally in the presence of an oxidising agent (e.g. air) or, alternatively, by treatment with an oxidising agent such as 2,3-dichloro-5,6-dicyano-1,4-benzoquinone (DDQ).

Compounds of formula V may be prepared by reaction of a compound of formula III as hereinbefore defined with a compound of formula XIV,
H₂NR² XIV
wherein R² is as hereinbefore defined, for example under conditions such as those described hereinbefore in respect of process step (ii) above.

Compounds of formula VII may be prepared by reaction of a compound of formula IV as hereinbefore defined with either:
(I) a compound of formula XV, 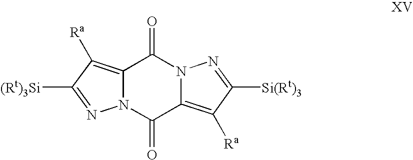
wherein R^{a} and R^{t} are as hereinbefore defined; or
(II) a compound of formula XIII (or a N-protected and/or O-protected (e.g. ester) derivative thereof) in which X represents Si(R^{t})₃, wherein R^{t} is as hereinbefore defined, for example under coupling conditions similar to those described hereinbefore in respect of process step (ii) above.

Compounds of formulae VIII and XV may be prepared from compounds of formula III, and compounds of formula XIII in which X represents Si(R^{t})₃, respectively, under dimerising conditions, for example in the presence of thionyl chloride (optionally in the presence of a suitable solvent and catalyst, such as one hereinbefore defined in respect of process step (ii)) at reflux. Other dimerising reagents include carbodiimides, such as 1,3-dicyclohexylcarbodiimide or 1-(3-dimethylaminopropyl)-3-ethylcarbodiimide (EDCI, or hydrochloride thereof) optionally in the presence of a suitable base (e.g. 4-dimethylaminopyridine).

Compounds of formula XIII (or derivatives thereof) in which R^{a} represents H or R^{c} as hereinbefore defined may be prepared by reaction of a compound of formula XVI,
X [symbol] R^{e} XVI
wherein R^{e} represents H or R^{c} and R^{c} and X are as hereinbefore defined, with a compound of formula XVII,
N₂—C(H)—C(O)OH XVII
or a O-protected (e.g. ester) derivative thereof, for example at elevated temperature (e.g. at between 80 and 120° C.) for between 1 and 3 days, optionally in the presence of an inert gas and preferably without the presence of solvent.

Compounds of formulae II, VI, IX, IXA, IXB, IXC, X, XI, XII, XIIIA, XIV, XVI and XVII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents B¹, R¹ and R² as hereinbefore defined may be modified one or more times, after or during the processes described above for preparation of compounds of formula I by way of methods that are well known to those skilled in the art. Examples of such methods include substitutions, reductions, oxidations, alkylations, hydrolyses, esterifications, and etherifications. Further, these reactions may occur concomitantly, for example, reduction of a nitro group to an amino group may occur at the same time as reduction of a C—Br bond to a C—H bond. The precursor groups can be changed to a different such group, or to the groups defined in formula I, at any time during the reaction sequence. In the case where R^{a} or R^{b} represents a halo group, such halo groups may be converted to another halo group one or more times, after or during the processes described above for the preparation of compounds of formula I. Appropriate reagents include NiCl₂ (for the conversion to a chloro group) or NiBr₂ (for the conversion to a bromo group). In this respect, the skilled person may also refer to “Comprehensive Organic Functional Group Transformations” by A. R. Katritzky, O. Meth-Cohn and C. W. Rees, Pergamon Press, 1995.

The synthesis of the bicycle R¹ may be performed at any point during the reaction sequence in accordance with standard heterocyclic chemistry. In this respect, the skilled person may refer to a standard heterocyclic chemistry textbook (e.g. “Heterocyclic Chemistry” by J. A. Joule, K. Mills and G. F. Smith, 3^{rd} edition, published by Chapman & Hall or “Comprehensive Heterocyclic Chemistry II” by A. R. Katritzky, C. W. Rees and E. F. V. Scriven, Pergamon Press, 1996). For example, the syntheses of a compound of formula IV in which the bicycle is a quinoxalinyl or quinazolinyl group, these bicycles may be prepared in accordance with the procedures described hereinafter.

Compounds of the formula I may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen or (when R^{a} and/or R^{b} represent —N(R^{3q})R^{3r}) the nitrogen of the —N(R^{3q})R^{3r} group may need to be protected. Suitable nitrogen-protecting groups include those which form:

(i) carbamate groups (i.e. alkoxy- or aryloxy-carbonyl groups);

(ii) amide groups (e.g. acetyl groups);

(iii) N-alkyl groups (e.g. hydroxymethyl or, preferably, benzyl groups);

(iv) N-sulfonyl groups (e.g. N-arylsulfonyl groups);

(v) N-phosphinyl and N-phosphoryl groups (e.g. diarylphosphinyl and diarylphosphoryl groups); or

(vi) N-silyl group (e.g. a N-trimethylsilyl group).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

Medical and Pharmaceutical Uses

Compounds of the invention are useful because they possess pharmacological activity. Such compounds are therefore indicated as pharmaceuticals. According to a further aspect of the invention there is provided a compound of formula I, as hereinbefore defined, or a pharmaceutically-acceptable salt thereof, but without provisos (A) to (D) and (F) for use as a pharmaceutical and/or in isolated (i.e. ex vivo) form.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I, as hereinbefore defined but without the provisos, or a pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined, or a pharmaceutically-acceptable salt thereof, but without provisos (A) to (D) and (F) in admixture with a pharmaceutically acceptable adjuvant, diluent or carrier.

Compounds of the invention may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of formula I, as hereinbefore defined but without the provisos, or a pharmaceutically-acceptable salt thereof, and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined but without the provisos, or a pharmaceutically-acceptable salt thereof, another therapeutic agent that is useful in the treatment of inflammation, and a pharmaceutically-acceptable adjuvant, diluent or carrier; and

(2) a kit of parts comprising components:

(a) a pharmaceutical formulation including a compound of formula I, as hereinbefore defined but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);

b) inhibitor (i.e. compound) or vehicle (0.5 μl DMSO);

c) 10 μL of a 10× concentrated solution of 15-lipoxygenase in PBS. The plates are incubated for 5 minutes at room temperature;

d) 5 μl of 0.125 mM arachidonic acid in PBS. The plate is then incubated for 10 minutes at room temperature;

e) the enzymatic reaction is terminated by the addition of 100 μl methanol; and

f) the amount of 15-hydroperoxy-eicosatetraenoic acid or 15-hydroxy-eicosatetraenoic acid is measured by reverse phase HPLC.

### EXAMPLES

The invention is illustrated by way of the following examples, in which the following abbreviations may be employed:
EDCI 1-(3-dimethylaminopropyl)-3-ethylcarbodiimide hydrochloride
DMAP 4-dimethylaminopyridine
DMF dimethylformamide
DMSO dimethylsulfoxide
EtOAc ethyl acetate
HATU O-(7-azabenzotriazol-1-yl)-N,N,N′,N′-tetramethyluronium hexafluorophosphate
MS mass spectrum
NMR nuclear magnetic resonance
rt room temperature
TBAF tetrabutylammonium fluoride
TBTU O-benzotriazol-1-yl-N,N,N′N′-tetramethyluronium tetrafluoroborate
THF tetrahydrofuran

Starting materials and chemical reagents specified in the synthesis described below are commercially available from, e.g. Sigma-Aldrich Fine Chemicals.

Unless otherwise stated, one or more tautomeric forms of compounds of the examples described hereinafter may be prepared in situ and/or isolated. All tautomeric forms of compounds of the examples described hereinafter should be considered to be disclosed.

Synthesis of Intermediates

### (i) Dipyrazolo[1,5-a;1′,5′-d]pyrazine-4,9-dione

DMF (0.1 mL, 1.4 mmol) was added dropwise to a stirred suspension of pyrazole-3-carboxylic acid (5.0 g, 44.6 mmol) in SOCl₂ (40 mL). The mixture was heated at reflux for 48 h, cooled and concentrated to give the sub-title compound as a white solid which was used without further purification.

### (ii) 2,7-Dimethyldipyrazolo[1,5-a;1′,5′-d]pyrazine-4,9-dione

### (a) 5-Methylpyrazole-3-carboxylic acid ethyl ester

The title compound is commercially available (Maybridge), but has been prepared as follows (J. Med. Chem. 2002, 45, 1035): Hydrazine monohydrate (25.6 g, 162 mmol) was added to a solution of ethyl 2,4-dioxovalerate (7.85 mL, 162 mmol) in absolute EtOH. The mixture was heated at reflux for 2 h and concentrated to give a yellow oil. Crystallisation from EtOH:water (1:3) gave the title compound (14.2 g, 57%) as colourless needles.

¹H NMR (DMSO-d₆, 400 MHz) δ 13.2 (br. s, 1H), 6.47 (s, 1H), 4.22 (q, 2H), 2.23 (s, 3H), 1.25 (t, 3H).

### (b) 5-Methylpyrazole-3-carboxylic acid

To a solution of 5-methylpyrazole-3-carboxylic acid ethyl ester (4.94 g, 32.0 mmol) in abs. EtOH (80 mL) was added NaOH (6.4 g, 160 mmol). The mixture was heated at reflux for 1 h and cooled to 20° C. The mixture was acidified with HCl (aq., 2 M, 85 mL, 170 mmol) and the pH adjusted to 3 with NaOH (aq., 2 M). The mixture was extracted with EtOAc (200 mL). The organic phase washed with NaCl (aq, sat, 50 mL) and concentrated to give the title compound (3.54 g, 88%) as a white solid.

¹H NMR (DMSO-d₆, 400 MHz) δ 12.83 (br. s, 1H), 6.43 (s, 1H), 2.22 (s, 3H).

### (c) 2,7-Dimethyldipyrazolo[1,5-a;1′,5′-d]pyrazine-4,9-dione

SOCl₂ (2.2 mL, 33.8 mmol) was added to a suspension of 5-methylpyrazole-3-carboxylic acid (1.44 g, 11.4 mmol) in toluene (10 mL) and the mixture was heated at reflux for 1 h. Concentration gave the sub-title product as a yellow solid, which was used without further purification.

### (iii) 5-Chloropyrazole-3-carboxylic acid

The intermediate may be synthesised by two alternative methods:

Method A

### (a) 1-Benzenesulfonyl-3-methylpyrazole

A mixture of 3-methylpyrazole (5 g, 60.9 mmol), benzenesulfonyl chloride (8.55 mL, 67 mmol), triethylamine (9.3 mL, 67 mmol) and acetonitrile was heated at reflux for 2 h, allowed to cool and concentrated. EtOAc (300 mL) was added and the solution was filtered and concentrated to provide a solid which was recrystallised from EtOAc to give the title compound as an off-white powder (Yield: 7.92 g, 58%).

¹H-NMR (DMSO-d₆): δ 8.35 (d, 1H), 7.97-7.94 (m, 2H), 7.78 (tt, 1H), 7.66 (t, 2H), 6.43 (d, 1H), 2.17 (s, 3H).

### (b) 5-Chloro-1-(2-chlorobenzenesulfonyl)-3-methylpyrazole

BuLi (1.6M, 9.45 mmol) was added under argon to a solution of 1-benzenesulfonyl-3-methylpyrazole (4.5 mmol; see step (a) above) in THF (50 mL) at −78° C. The mixture was stirred for approximately 30 min before hexachloroethane (3.7 g, 15.8 mmol) was added. After stirring at −78° C., NH₄Cl (aq, sat, 50 mL) was added and the mixture was allowed to come to rome temperature. Water (50 mL) was added, the layers separated, and the aqueous phase extracted with EtOAc (2×100 mL). The combined organic phases were dried (Na₂SO₄) and concentrated. Purification by chromatography (1:4 EtOAc/heptane) followed by recrystallisation from EtOAc/heptane gave the title compound as white crystals (Yield: 1.1 g, 84%).

¹H-NMR (DMSO-d₆): δ 8.17 (dd, 1H), 7.87-7.67 (m, 4H), 2.15 (s, 3H).

### (c) 5-Chloro-3-methylpyrazole

Sodium ethoxide (2.5M, 16.1 mL, 40.3 mmol) was added to a stirred solution of 5chloro-1-(2-chlorobenzenesulfonyl)-3-methylpyrazole (6.9 g, 27 mmol; see step (b) above) in EtOH (50 mL). After 30 min at rt, water (100 mL) was added and the mixture was neutralised with HCl (aq, 2M) and extracted with EtOAc (3×100 mL). Concentration of the combined extracts resulted in precipitation prior to complete solvent removal. The precipitate was filtered off and the filtrate was concentrated to give a brown oil that crystallised on standing (Yield: 1.0, g 33% which was used without further purification.

¹H-NMR (DMSO-d₆): δ 12.66 (bs, 1H), 6.03 (d, 1H), 2.20 (s, 3H).

### (d) 5-Chloropyrazole-3-carboxylic acid

A solution of KMnO₄ (3.5 g, 22 mmol) in water (120 mL) was added in portions over a period of 5 h at 70° C. to a solution of 5-chloro-3-methylpyrazole (1.0 g, 8.8 mmol; see step (c) above) in water (50 mL) and tert-butanol (1 mL). The mixture was stirred at 70° C. overnight and filtered through Celite®. The colourless filtrate was concentrated and acidified with HCl (2M). Filtration gave the title compound as a white powder which was used without further purification. (Yield: 913 mg, 80%).

¹H-NMR (DMSO-d₆): δ 13.65 (br s, 1H), 6.80 (s, 1H), 4.40 (bs, 1H).

Method B

### (a) 5-Chloro-3-methylpyrazole

A mixture of 5-chloro-1,3-dimethylpyrazole (0.34 g, 2.6 mmol) and pyridine hydrochloride (1.51 g, 13.1 mmol) in a sealed vessel was heated using microwave irradiation for 2 h at 200° C. After cooling to rt, EtOAc (15 mL) was added and the mixture washed with HCl (2M, 10 mL), NaCl (aq, sat), dried (MgSO₄) and concentrated affording the sub-title compound as a white solid (Yield: 210 mg (67%)).

MS (M⁺+H) m/z=117

¹H-NMR (DMSO-d₆, 400 MHz), δ 12.66 (br s, 1H), 6.03 (m, 1H), 2.19 (s, 3H).

### (b) 5-Chloropyrazole-3-carboxylic acid

A mixture of 5-chloro-3-methylpyrazole (3.6 mmol; see step (a) above), water (6 mL), tert-butanol (1.2 mL) and KMnO₄ (1.42 g, 9 mmol) was stirred at 75° C. overnight. The hot mixture was filtered and the solids washed with boiling water. The combined filtrates were extracted twice with EtOAc. The combined extracts were washed with NaCl (aq, sat), dried (MgSO₄) and concentrated to provide a solid, which was crystallised from EtOAc/hexane/pentane to give the sub-title product as white crystals (Yield: 350 mg (67%)).

¹H-NMR (DMSO-d₆, 400 MHz), δ 13.65 (br s, 1H), 6.80 (s, 1H), 4.40 (bs, 1H).

### (iv) 4-Bromopyrazole-3-carboxylic acid

Bromine (800 mg, 5.0 mmol) was added to a solution of pyrazole-3-carboxylic acid (500 mg, 4.5 mmol) in acetic acid (20 mL). The mixture was stirred at rtrt for 18 h followed by addition of water (100 mL) and extraction with diethyl ether (3×30 mL). The combined organic phases were washed with water (50 mL) and concentrated to give the title product (750 mg, 87%) as a pale yellow solid.

¹H NMR (DMSO-d₆, 400 MHz) δ 7.92 (s, 1H).

### (v) 5-Trifluoromethylpyrazole-3-carboxylic acid

### (a) 1,1,1-Trifluoro-4-methoxypent-3-en-2-one

A mixture of 2-methoxypropene (7.7 g, 132 mmol) and pyridine (9.7 mL, 120 mmol) was added dropwise to trifluoroacetic anhydride (25.2 g, 120 mmol) while cooled at −30° C. Diethyl ether (50 mL) was added and the mixture was left for 18 h at room temperature. Filtration and concentration gave a yellow oil that was taken up in CH₂Cl₂. The mixture washed with HCl (0.1 M, aq, 50 mL), water (50 mL), dried (Na₂SO₄) and concentrated affording 23 g of an orange oil which was used in the following step without any further purification.

¹H NMR (DMSO-d₆, 400 MHz) δ 5.68 (s, 1H), 3.80 (s, 3H), 2.41 (s, 3H).

### (b) 3-Methyl-5-trifluoromethylpyrazole

Hydrazine hydrate (4.0 g, 79 mmol) was added dropwise to a solution of 1,1,1-trifluoro-4-methoxypent-3-en-2-one (10.0 g, 59 mmol) in EtOH (30 mL). The mixture was heated at reflux for 2 h, cooled and concentrated. The residue was taken up in diisopropyl ether and dried (Na₂SO₄). Concentration gave the sub-title compound that was used in the following step without further purification. Yield: 7.0 g (79%).

¹H NMR(DMSO-d₆, 400 MHz) δ 6.15 (s, 1H), 2.29 (s, 3H).

### (c) 5-Trifluoromethylpyrazole-3-carboxylic acid

A mixture of 3-methyl-5-trifluoromethylpyrazole (3.0 g, 20 mmol), KMnO₄ (3.0 g, 19.0 mmol) and water (80 mL) was heated at 80° C. for 18 h. The mixture was filtered through Celite®. The filtrate was acidified with 2M HCl (aq) and extracted with diethyl ether (3×50 mL). The combined extracts were dried (Na₂SO₄) and concentrated to give the title compound as yellow crystals which were used without further purification. Yield: 1.6 g (44%).

Synthesis of Bicyclic Arylamines

Bicyclic arylamines which were not available commercially were synthesised in accordance with procedures know to those skilled in the art, for example, such as those described hereinafter.

3-Amino-2-methylquinoline, 3-amino-6,8-dibromo-2-methylquinoline and 3-amino-6-chloro-2-methylquinoline were prepared in accordance with the literature procedure (Wang, Y. D. et al. Tetrahedron 2004, 60, 2937-2942). 3-Amino-6,7-dihydro-5H-cyclopenta[b]pyridine, 3-amino-5,6,7,8-tetrahydroquinoline, 3-amino-6,7,8,9-tetrahydro-5H-cyclohepta[b]pyridine and 3-amino-7,8-dihydro-5H-pyrano[4,3-b]pyridine were prepared in accordance with the literature procedures (Takada, S. et al. J. Med. Chem. 1996, 39, 2844-2851; Tohda, Y. et al. Bull. Chem. Soc. Japan 1990, 63, 2820-2827). 3-Amino-5,6,7,8-tetrahydro[1,6]naphthyridine-6-carboxylic acid tert-butyl ester and 3-amino-6-methyl-5,6,7,8-tetrahydro[1,6]naphthyridine where prepared in accordance with the literature procedure (Harling, J. D. et al. Synthetic Communications, 2001, 31, 787-797). 5-Amino-3-azatricyclo-[6.2.1.0^{2,7}]undeca-2(7),3,5-triene was prepared in accordance with international patent application WO 98/46572 (Example 22). 3-Amino-7-methoxyquinoline, 3-amino-5-methoxyquinoline, 3-amino-6,7-dimethoxyquinoline, 8-amino-2,3-dihydro[1,4]dioxano[2,3-g]quinoline and 7-amino-[1,3]dioxolo[4,5-g]quinoline were prepared by a procedure analogous to a literature for 3-amino-5,6,7,8-tetrahydroquinoline (Takada, S. et al. J. Med. Chem. 1996, 39, 2844-2851) employing Pd-catalysed hydrogenation of the corresponding nitro compounds. 7-Methoxy-3-nitroquinoline was prepared in accordance with a literature procedure (Krasavin I. A. et al. Metody Polucheniya Khimicheskih Reaktivov i Preparatov, Moscow 1971, 23, 94-96). The other relevant nitro compounds were synthesised analogously (see also Morley J. S. and Simpson J. C. E. J. Chem. Soc. 1948, 2024-2027).

### 3-Amino-6-tert-butyl-5,6,7,8-tetrahydroquinoline

The title compound was prepared in accordance with a literature procedure described for 3-amino-5,6,7,8-tetrahydroquinoline (Takada, S. et al. J. Med. Chem. 1996, 39, 2844-2851) employing 4-tert-butylcyclohexanone. The compound was isolated in 49% yield as an off-white solid.

¹H NMR (DMSO-d₆, 400 MHz) δ 7.68 (d, 1H), 6.62 (d, 1H), 5.09 (br. s, 2H), 2.77-2.35 (m, 4H), 1.97-1.93 (m, 1H), 1.40-1.22 (m, 2H), 0.90 (s, 9H).

### 3-Amino-6-hydroxy-5,6,7,8-tetrahydroquinoline

### (a) 3-Nitro-7,8-dihydro-5′H-spiro[1,3]dioxolane-6,6′-quinoline

The sub-title compound was prepared in accordance with a literature procedure described for 3-amino-5,6,7,8-tetrahydroquinoline (Takada, S. et al. J. Med. Chem. 1996, 39, 2844-2851) from 1,4-dioxaspiro[4,5]decan-8-one. The compound was isolated in 83% yield as slightly orange crystals.

¹H NMR (DMSO-d₆, 400 MHz) δ 9.12 (d, 1H), 8.33 (d, 1H), 3.97 (s, 4H), 3.12-3.07 (m, 4H), 2.03 (t, 2H).

### (b) 3-Nitro-7,8-dihydro-5H-quinolin-6-one

A mixture of 3-nitro-7,8-dihydro-5′H-spiro[1,3]dioxolane-6,6′-quinoline (737 mg, 3.12 mmol), HCl (aq, 2M, 6.6 mL), water (3.3 mL) and acetone (10 mL) was heated at reflux for 45 min. The mixture was cooled to rt, neutralised with NaHCO₃ (aq, sat, 50 mL) and extracted with EtOAc (3×20 mL). The combined organic phases were dried (Na₂SO₄), stirred with activated charcoal (200 mg) for 30 min and filtered first through Celite® and then through silica gel. Concentration gave the sub-title compound (416 mg, 69%) as a yellow solid. According to the ¹H NMR the compound exists as a mixture (˜7:3) of the corresponding keto and enol forms.

¹H NMR (DMSO-d₆, 400 MHz) δ 10.12 (s, 0.3H), 9.18 (d, 0.7H), 8.79 (d, 0.3H), 8.45 (d, 0.7H), 7.97 (d, 0.3H), 5.69 (s, 0.3H), 3.83 (s, 1.4H), 3.30 (t, 1.4H), 3.06 (t, 0.7H), 2.60 (t, 1.4H), 2.51 (t, 0.7H).

### (c) 6-Hydroxy-3-nitro-5,6,7,8-tetrahydroquinoline

NaBH₄ (173 mg, 4.58 mmol) was added to a solution of 3-nitro-7,8-dihydro-5H-quinolin-6-one (220 mg, 1.14 mmol) in MeOH (50 mL) at −43° C. under argon. After 2 h of stirring the reaction was quenched with crushed ice and the temperature was allowed to rise to rt The mixture was concentrated and the residue extracted with CH₂Cl₂ (3×10 mL). The combined organic phases were dried (Na₂SO₄) and concentrated to give the sub-title compound (170 mg, 76%) as a yellow solid.

¹H NMR (DMSO-d₆, 400 MHz) δ 9.09 (d, 1H), 8.32 (d, 1H), 4.95 (d, 1H), 4.10-4.00(m, 1H), 3.11-2.73 (m, 4H), 2.01-1.80 (m, 2H).

### (d) 3-Amino-6-hydroxy-5,6,7,8-tetrahydroquinoline

A mixture of 6-hydroxy-3-nitro-5,6,7,8-tetrahydroquinoline (156 mg, 0.803 mmol) and palladium on active carbon (10% Pd, 75 mg) in MeOH (10 mL) was hydrogenated at ambient temperature and pressure for 30 min. The mixture was filtered through Celite® and concentrated to give the title compound (131 mg, 99%) as a yellow solid.

¹H NMR (DMSO-d₆, 400 MHz) 7.68 (d, 1H), 6.60 (d, 1H), 5.2-4.8 (br. s, 2H), 4.73 (d, 1H), 3.93-3.80 (m, 1H), 2.84-2.44 (m, 4H), 1.93-1.82 (m, 1H), 1.73-1.60 (m, 1H).

### 3-Amino-6,6-dioxo-5,6,7,8-tetrahydro-6λ⁶-thiopyrano[4,3-b]pyridine

### (a) 3-Nitro-7,8-dihydro-5H-thiopyrano[4,3-b]pyridine

The sub-title compound was prepared in accordance with a procedure described in international patent application WO 98/46572 (Example 25).

### (b) 3-Nitro-7,8-dihydro-5H-thiopyrano[4,3-b]pyridine 6,6-dioxide

A mixture of 3-nitro-7,8-dihydro-5H-thiopyrano[4,3-b]pyridine (127 mg, 0.65 mmol), 3-chloroperbenzoic acid (335 mg, 1.94 mmol) and CH₂Cl₂ (10 mL) was stirred at rt for 18 h. A solution of Na₂SO₃ (245 mg, 1.94 mmol) in water (5 mL) was added and the mixture was stirred at rt for 5 min. The layers were separated and the organic phase washed with Na₂CO₃ (aq, 1M, 5 mL), dried (Na₂SO₄) and concentrated. The crude material was purified by chromatography (EtOAc:heptane, 2:1) to give the title product (55 mg, 37%) as a white solid.

¹H NMR (DMSO-d₆, 400 MHz) δ 9.24 (d, 1H), 8.49 (d, 1H), 4.73 (s, 2H), 3.67-3.62 (m, 2H), 3.53 (t, 2H).

### (c) 3-Amino-6,6-dioxo-5,6,7,8-tetrahydro-6λ⁶-thiopyrano[4,3-b]pyridine

3-Nitro-7,8-dihydro-5H-thiopyrano[4,3-b]pyridine 6,6-dioxide (53 mg, 0.232 mmol) in MeOH:CH₂Cl₂ (1:1, 10 mL) was hydrogenated at ambient temperature and pressure over palladium on active carbon (10% Pd, 50 mg) for 30 min. The mixture was filtered through Celite® and concentrated to give the title compound (45 mg, 98%) as a white solid.

¹H NMR (DMSO-d₆, 400 MHz) δ 7.82 (d, 1H), 6.69 (d, 1H), 6.0-5.0 (br. s, 2H), 4.40 (s, 2H), 3.44 (t, 2H), 3.20 (t, 2H).

### 6-Acetyl-3-amino-5,6,7,8-tetrahydro[1,6]naphthyridine

### (a) 3-Nitro-7,8-dihydro-5H-[1,6]naphthyridine-6-carboxylic acid tert-butyl ester

The sub-title compound was prepared in accordance with a literature procedure (Harling, J. D. et al Synthetic Communications, 2001, 31, 787-797).

### (b) 3-Nitro-5,6,7,8-tetrahydro[1,6]naphthyridin-6-ium trifluoroacetate

Trifluoroacetic acid (5 mL) was added dropwise to a solution of 3-nitro-7,8-dihydro-5H-[1,6]naphthyridine-6-carboxylic acid tert-butyl ester (283 mg, 1.01 mmol) in CH₂Cl₂ (25 mL) and the mixture was stirred for 75 min at rt Concentration gave an orange oil which was triturated with diethyl ether (25 mL). The solid was filtered off and recrystallised from EtOAc/MeOH to give the title compound (189 mg, 64%) as white crystals.

¹H NMR (DMSO-d₆, 400 MHz) δ 9.38 (s, 2H), 9.24 (d, 1H), 8.60 (d, 1H), 4.47 (s, 2H), 3.55 (t, 2H), 3.21 (t, 2H).

### (c) 6-Acetyl-3-nitro-5,6,7,8-tetrahydro[1,6]naphthyridine

Acetic anhydride (0.191 mL, 2.03 mmol) was added dropwise to a solution of 3-nitro-5,6,7,8-tetrahydro[1,6]naphthyridin-6-ium trifluoroacetate (119 mg, 0.406 mmol) in pyridine (5 mL). The mixture was stirred at rt for 30 min and concentrated. The residue was crystallised from EtOAc to give the sub-title compound (57 mg, 63%) as white needles. According to ¹H NMR the compound exists as a mixture (˜2:3) of rotamers.

¹H NMR (DMSO-d₆, 400 MHz) δ 9.18-9.14 (m, 1H), 8.53 (d, 0.6H), 8.48 (d, 0.4H), 4.83 (s, 0.5H), 4.76 (s, 1.2H), 3.79 (t, 2H), 3.10 (t, 1.2H), 2.97 (t, 0.8H), 2.12 (s, 1.8H), 2.10 (s, 1.2H).

### (d) 6-Acetyl-3-amino-5,6,7,8-tetrahydro[1,6]naphthyridine

6-Acetyl-3-nitro-5,6,7,8-tetrahydro[1,6]naphthyridine (52 mg, 0.235 mmol) in MeOH (10 mL) was hydrogenated at ambient temperature and pressure over palladium on active carbon (10% Pd, 50 mg) for 30 min. The mixture was filtered through Celite® and concentrated to give the title compound (45 mg, 100%) as a yellow oil. According to ¹H NMR the compound exists as a mixture (˜2:3) of the cis and trans amide forms.

¹H NMR (DMSO-d₆, 400 MHz) δ 7.74 (m, 1H), 6.69 (d, 1H), 5.50-5.00 (br. s, 2H), 4.52 (s, 0.8H), 4.47 (s, 1.2H), 3.70-3.63 (m, 2H), 2.75 (t, 1.2H), 2.63 (t, 0.8H), 2.07 (s, 1.8H), 2.06 (s, 1.2H).

### 4-Aminoisoquinoline

### (a) N-Benzyl-4-aminoisoquinoline

A mixture of 4-bromoisoquinoline (1.00 g, 4.81 mmol), benzylamine (620 mg, 5.77 mmol), Cs₂CO₃ (7.83 g, 24.0 mmol), tris(dibenzylideneacetone)di-palladium(0) (132 mg, 0.144 mmol) and 2,2′-bis(diphenylphosphino)-1,1′-binaphthyl (90 mg, 0.144 mmol) in dry toluene (40 mL) was stirred under argon at 90° C. for 18 h. After cooling to rt the precipitate was filtered off and washed with EtOAc. The combined liquids were concentrated and the residue purified by chromatography (EtOAc:heptane) to give the sub-title compound (1.00 g, 89%) as a yellow solid.

MS (M⁺+H) m/z 235.

¹H NMR (DMSO-d₆, 400 MHz) δ 8.49 (s, 1H), 8.27 (d, 1H), 7.92 (d, 1H), 7.68 (t, 1H), 7.62-7.56 (m, 2H), 7.41 (d, 2H), 7.30 (t, 2H) 7.21 (t, 1H) 7.03 (t, 1H), 4.52 (d, 2H).

### (b) 4-Aminoisoquinoline

A mixture of N-benzyl-4-aminoisoquinoline (1.00 g, 4.27 mmol), acetic acid (30 mL) and H₂SO₄ (conc, 7.5 mL) was stirred at 100° C. for 6 hours. After cooling to rt the reaction was quenched with sat. aq. Na₂CO₃ (200 mL) and extracted with EtOAc (5×25 mL). The combined organic phases were concentrated and the residue was purified by chromatography (EtOAc) to give the title compound (325 mg, 53%) as a yellow solid.

¹H NMR (DMSO-d₆, 400 MHz) δ 8.49 (s, 1H), 8.10 (d, 1H), 7.90 (d, 1H), 7.87 (s, 1H), 7.64-7.54 (m, 2H), 5.82 (s, 2H).

### 8-Aminoisoquinoline

### (a) 5-Bromo-8-nitroisoquinoline

N-Bromosuccinimide (17.80 g, 100 mmol) was added in portions over 15 min to a solution of isoquinoline (11.00 g, 85 mmol) in H₂SO₄ (conc, 100 mL) cooled to −25° C. After stirring at rt for 22 h, KNO₃ (11.12 g, 110 mmol) was added and the mixture was stirred for 70 min. The mixture was poured onto crushed ice and neutralised with NH₃ (aq, sat, 100 mL). The precipitate was filtered off, washed with water and dried. Crystallisation from MeOH gave the sub-title product (9.27 g, 43%) as a dark brown solid.

¹H NMR (DMSO-d₆, 400 MHz) δ 10.01 (s, 1H), 8.84 (d, 1H), 8.19 (d, 1H), 8.15 (d, 1H), 8.11 (d, 1H).

### (b) 8-Aminoisoquinoline

5-Bromo-8-nitroisoquinoline (1.00 g, 3.95 mmol) in MeOH (70 mL) was hydrogenated at ambient temperature and pressure over palladium on active carbon (10% Pd, 200 mg) for 18 h. The mixture was filtered through Celite® and concentrated. The residue was purified by chromatography (EtOAc:heptane) to give the title compound (100 mg, 18%) as a green-brown solid.

¹H NMR (DMSO-d₆, 400 MHz) δ 9.43 (s, 1H), 8.32 (d, 1H), 7.54 (d, 1H), 7.40 (dd, 1H), 6.99 (d, 1H), 6.72 (d, 1H), 6.22 (s, 2H).

### 5-Aminoquinoxaline

### (a) 5-Nitroquinoxaline

Glyoxal (aq, 8.8 M, 2.6 mL, 22.8 mmol) was added to a mixture of 3-nitro-benzene-1,2-diamine (1.163 g, 7.59 mmol) in EtOH (16 mL). The mixture was heated at reflux for 2 h and stirred at rt for 3 days. Water (50 mL) was added and the resulting mixture was extracted with CH₂Cl₂ (3×20 mL). The combined extracts were dried (MgSO₄) and concentrated to give an orange-red solid which was recrystallised from water to give the sub-title compound (439 mg, 33%) as pale yellow needles.

¹H NMR (CDCl₃, 400 MHz) δ 9.05 (d, 1H), 9.01 (d, 1H), 8.38 (dd, 1H), 8.20 (dd, 1H), 7.89 (t, 1H).

### (b) 5-Aminoquinoxaline

A mixture of 5-nitroquinoxaline (439 mg, 2.51 mmol), ammonium formate (158 mg, 2.51 mmol) and palladium on active carbon (10% Pd, 50 mg) in MeOH (25 mL) was stirred in a sealed vial at 100° C. for 18 h. The mixture was cooled to rt, filtered through Celite® and concentrated to give a brown solid. Chromatography (EtOAc:heptane, 1:2) gave the title compound (203 mg, 56%) as a brown-yellow solid.

¹H NMR (DMSO-d₆, 400 MHz) δ 8.85 (d, 1H), 8.72 (d, 1H), 7.53 (t, 1H), 7.17 (dd, 1H), 6.92 (dd, 1H), 6.14 (br. s, 2H).

### Examples 1 to 40

### General Procedure

The relevant starting material (i.e. (i) or (ii) above; 0.40 mmol) and the relevant bicyclic arylamine (0.64 mmol) were dissolved in pyridine:DMF mixture (9:1, 3.0 mL) and the mixture was heated at the indicated temperature for the indicated period of time. The volatiles were removed in vacuo and the residue was purified by chromatography using EtOAc/heptane as an eluent.

TABLE 1
Examples (Ex.) 1 to 40

| | | | | Reaction | | |
|---|---|---|---|---|---|---|
| | | Prepared from | | conditions | | |
| | | (intermediate (i) or (ii) | | | Temp. | |
| Ex. | Chemical name | and arylamine) | | Time h | ° C. | Yield % |
| 1 | Pyrazole-3-carboxylic acid | i | 5-Aminoiso- | 18 | 105 | 30 |
| | isoquinolin-5-ylamide | | quinoline | | | |
| 2 | Pyrazole-3-carboxylic acid | i | 2-Amino- | 18 | 105 | 40 |
| | quinolin-2-ylamide | | quinoline | | | |
| 3 | Pyrazole-3-carboxylic acid | i | 3-Aminoiso- | 18 | 105 | 11 |
| | isoquinolin-3-ylamide | | quinoline | | | |
| 4 | Pyrazole-3-carboxylic acid | i | 3-Amino- | 18 | 105 | 54 |
| | quinolin-3-ylamide | | quinoline | | | |
| 5 | Pyrazole-3-carboxylic acid | i | 6-Amino- | 18 | 105 | 40 |
| | quinolin-6-ylamide | | quinoline | | | |
| 6 | Pyrazole-3-carboxylic acid | i | 5-Amino- | 18 | 105 | 24 |
| | quinolin-5-ylamide | | quinoline | | | |
| 7 | 5-Methylpyrazole-3- | ii | 5-Amino- | 18 | 105 | 17 |
| | carboxylic acid quinolin-5- | | quinoline | | | |
| | ylamide | | | | | |
| 8 | 5-Methylpyrazole-3- | ii | 3,4-(Difluoromethylenedioxy)- | 18 | 105 | 42 |
| | carboxylic acid (3,4- | | | | | |
| | (difluoromethylenedioxy)- | | aniline | | | |
| | phenyl)amide | | | | | |
| 9 | Pyrazole-3-carboxylic acid | i | 4-Aminoindole | 5 | 80 | 67 |
| | indol-4-ylamide | | | | | |
| 10 | Pyrazole-3-carboxylic acid | i | 5-Amino-3- | 18 | 105 | 6 |
| | (3-methylcinnolin-5- | | methylcinnoline | | | |
| | yl)amide | | | | | |
| 11 | Pyrazole-3-carboxylic acid | i | 6-Amino-1,2- | 18 | 105 | 1 |
| | (1,4-benzodioxan-6-yl)- | | benzo-dioxane | | | |
| | amide | | | | | |
| 12 | 5-Methylpyrazole-3- | ii | 8-Amino- | 18 | 105 | 38 |
| | carboxylic acid quinolin-8- | | quinoline | | | |
| | ylamide | | | | | |
| 13 | Pyrazole-3-carboxylic acid | i | 5-Aminoindole | 18 | 105 | 55 |
| | indol-5-ylamide | | | | | |
| 14 | Pyrazole-3-carboxylic acid | i | 6-Amino-2- | 18 | 105 | 34 |
| | (2-methylquinolin-6- | | methylquinoline | | | |
| | yl)amide | | | | | |
| 15 | Pyrazole-3-carboxylic acid | i | 1-Aminoiso- | 20 | 105 | 24 |
| | isoquinolin-1-ylamide | | quinoline | | | |
| 16 | 5-Methylpyrazole-3- | ii | 6-Amino-1,4- | 18 | 80 | 1 |
| | carboxylic acid (1,4- | | benzodioxane | | | |
| | benzodioxan-6-yl)amide | | | | | |
| 17 | Pyrazole-3-carboxylic acid | i | 5-Aminoindazole | 6 | 80 | 38 |
| | (indazol-5-yl)amide | | | | | |
| 18 | Pyrazole-3-carboxylic acid | i | 6-Amino-2- | 6 | 80 | 29 |
| | (2-mercaptobenzothiazol- | | mercaptobenzo- | | | |
| | 6-yl)amide | | thiazole | | | |
| 19 | Pyrazole-3-carboxylic acid | i | 5-Amino-2- | 6 | 80 | 1 |
| | (2-methylbenzothiazol-5- | | methylbenzo- | | | |
| | yl)amide | | thiazole | | | |
| 20 | Pyrazole-3-carboxylic acid | i | 4-Amino- | 5 | 80 | 22 |
| | benzo[2,1,3]thiadiazol-4- | | benzo[2,1,3]thia- | | | |
| | ylamide | | diazole | | | |
| 21 | 5-Methylpyrazole-3- | ii | 3-Amino- | 18 | 80 | 63 |
| | carboxylic acid quinolin-3- | | quinoline | | | |
| | ylamide | | | | | |
| 22 | Pyrazole-3-carboxylic acid | i | 4-Amino- | 8 | 80 | 18 |
| | quinolin-4-ylamide | | quinoline | | | |
| 23 | Pyrazole-3-carboxylic acid | i | 4-Amino- | 18 | 80 | 90 |
| | isoquinolin-4-ylamide | | isoquinoline | | | |
| 24 | Pyrazole-3-carboxylic acid | i | 3-Amino-7- | 18 | 80 | 64 |
| | (7-methoxyquinolin-3- | | methoxyquinoline | | | |
| | yl)amide | | | | | |
| 25 | Pyrazole-3-carboxylic acid | i | 4-Amino-2- | 18 | 80 | 13 |
| | (2-methylquinolin-4-yl)- | | methylquinoline | | | |
| | amide | | | | | |
| 26 | Pyrazole-3-carboxylic acid | i | 3-Amino-5- | 18 | 80 | 35 |
| | (5-methoxyquinolin-3- | | methoxyquinoline | | | |
| | yl)amide | | | | | |
| 27 | Pyrazole-3-carboxylic acid | i | 3-Amino-5,6,7,8- | 18 | 80 | 21 |
| | (5,6,7,8-tetrahydro- | | tetrahydro- | | | |
| | quinolin-3-yl)-amide | | quinoline | | | |
| 28 | Pyrazole-3-carboxylic acid | i | 3-Amino-6,7- | 4 | 80 | 29 |
| | (6,7-dihydro-5H- | | dihydro-5H- | | | |
| | cyclopenta[b]pyrid-3- | | cyclopenta[b]pyridine | | | |
| | yl)amide | | | | | |
| 29 | Pyrazole-3-carboxylic acid | i | 3-Amino-6,6- | 18 | 80 | 79 |
| | (6,6-dioxo-5,6,7,8- | | dioxo-5,6,7,8- | | | |
| | tetrahydro-6λ⁶- | | tetrahydro-6λ⁶- | | | |
| | thiopyrano[4,3-b]pyrid-3- | | thiopyrano[4,3- | | | |
| | yl)amide | | b]pyridine | | | |
| 30 | Pyrazole-3-carboxylic acid | i | 5-Amino-3- | 4 | 80 | 61 |
| | (3-azatricyclo- | | azatricyclo | | | |
| | [6.2.1.0^{2,7}]undeca-2(7),3,5- | | [6.2.1.0^{2,7}]undeca- | | | |
| | trien-5-yl)amide | | 2(7),3,5-triene | | | |
| 31 | Pyrazole-3-carboxylic acid | i | 3-Amino-6- | 3 | 80 | 83 |
| | (6-hydroxy-5,6,7,8- | | hydroxy-5,6,7,8- | | | |
| | tetrahydroquinolin-3- | | tetrahydro- | | | |
| | yl)amide | | quinoline | | | |
| 32 | Pyrazole-3-carboxylic acid | i | 3-Amino-6- | 6 | 80 | 73 |
| | (6-methyl-5,6,7,8- | | methyl-5,6,7,8- | | | |
| | tetrahydro[1,6]naphthyridin- | | tetrahydro[1,6]- | | | |
| | 3-yl)amide | | naphthyridine | | | |
| 33 | Pyrazole-3-carboxylic acid | i | 3-Amino-7,8- | 18 | 80 | 86 |
| | (7,8-dihydro-5H- | | dihydro-5H- | | | |
| | pyrano[4,3-b]pyrid-3- | | pyrano[4,3-b]- | | | |
| | yl)amide | | pyridine | | | |
| 34 | Pyrazole-3-carboxylic acid | i | 3-Amino-6-tert- | 48 | 80 | 68 |
| | (6-tert-butyl-5,6,7,8- | | butyl-5,6,7,8- | | | |
| | tetrahydroquinolin-3- | | tetrahydro | | | |
| | yl)amide | | quinoline | | | |
| 35 | 3-[(Pyrazole-3- | i | 3-Amino-5,6,7,8- | 2 | 80 | 40 |
| | carbonyl)amino]-5,6,7,8- | | tetrahydro- | | | |
| | tetrahydro[1,6]naphthyridine- | | [1,6]naphthyridine- | | | |
| | 6-carboxylic acid | | 6-carboxylic | | | |
| | tert-butyl ester | | acid tert-butyl | | | |
| | | | ester | | | |
| 36 | Pyrazole-3-carboxylic acid | i | 5-Aminoquinoxaline | 18 | 80 | 43 |
| | quinoxalin-5-ylamide | | | | | |
| 37 | Pyrazole-3-carboxylic acid | i | 5-Amino- | 18 | 80 | 11 |
| | benzoimidazol-5-ylamide | | benzoimidazole | | | |
| 38 | Pyrazole-3-carboxylic acid | i | 8-Aminoiso- | 72 | 80 | 26 |
| | isoquinolin-8-ylamide | | quinoline | | | |
| 39 | Pyrazole-3-carboxylic acid | i | 6-Acetyl-3- | 18 | 80 | 77 |
| | (6-acetyl-5,6,7,8- | | amino-5,6,7,8- | | | |
| | tetrahydro- | | tetrahydro- | | | |
| | [1,6]naphthyridin-3- | | [1,6]naphthyridine | | | |
| | yl)amide | | | | | |
| 40 | Pyrazole-3-carboxylic acid | i | 4-Amino-7- | 21 | 120 | 66 |
| | (7-chloroquinolin-4- | | chloroquinoline | | | |
| | yl)amide | | | | | |

TABLE 2
Physical properties of the compounds of Examples 1-40

| | | MS | |
|---|---|---|---|
| | | (M⁺+1), | |
| Ex. | M.W. | m/z | ¹H NMR(DMSO-d₆, 400MHz), δ |
| 1 | 238.24 | 239 | 13.48(1H, s), 10.28(1H, s), 9.35(1H, s), 8.53(1H, s), |
| | | | 8.03-7.94(3H, m), 7.76(1H, d), 7.71(1H, t), 6.83(1H, s) |
| 2 | 238.24 | 239 | 13.59(1H, s), 9.89(1H, s), 8.44-8.39(2H, m), 7.95(2H, d), |
| | | | 7.85(1H, d), 7.73(1H, dt), 7.51(1H, t), 6.92(1H, s) |
| 3 | 238.24 | 239 | 13.54(s, 1H), 9.70(s, 1H), 9.19(s, 1H), 8.57(s, 1H), 8.08(d, |
| | | | 1H), 7.95(d, 2H), 7.74(dt, 1H), 7.56(t, 1H), 6.89(s, 1H) |
| 4 | 238.24 | 239 | 13.51(1H, s), 10.57(1H, s), 9.22(1H, d), 8.85(1H, d), |
| | | | 7.98-7.92(3H, m), 7.65(1H, dt), 7.58(1H, t), 6.84(1H, d) |
| 5 | 238.24 | 239 | 13.47(1H, s), 10.38(1H, s), 8.79(1H, dd), 8.56(1H, s), |
| | | | 8.28(1H, d), 8.12(1H, dd), 7.97(1H, d), 7.93(1H, s), 7.48(1H, |
| | | | dd), 6.83(1H, d) |
| 6 | 238.24 | 239 | 13.46(1H, s), 10.32(1H, s), 8.92(1H, d), 8.33(1H, d), |
| | | | 7.94-7.92(2H, m), 7.80-7.74(2H, m), 7.55(1H, dd), 6.82(1H, s) |
| 7 | 252.27 | 253 | 13.13(1H, s), 10.22(1H, s), 8.92(1H, d), 8.31(1H, d), |
| | | | 7.92(1H, d), 7.79-7.74(2H, m), 7.55(1H, dd), 6.55(1H, s), |
| | | | 2.33(3H, s) |
| 8 | 281.22 | 282 | 13.15(1H, s), 10.07(1H, s), 7.35(1H, d), 7.19(2H, m), |
| | | | 6.52(1H, s), 2.30(3H, s) |
| 9 | 226.23 | 227 | 13.42(1H, br. s), 11.18(1H, br. s), 9.50(1H, br. s), 7.92(1H, |
| | | | br. s), 7.60(1H, br. s), 7.33(1H, t), 7.20(1H, d), 7.07(1H, t), |
| | | | 6.81(1H, br. s), 6.49(1H, br. s) |
| 10 | 253.26 | 254 | 13.50(s, 1H), 10.40(s, 1H), 8.31(d, 1H), 7.96-7.86(m, 4H), |
| | | | 6.84(s, 1H), 2.88(s, 3H) |
| 11 | 245.23 | 246 | 13.01(s, 1H), 9.76(s, 1H), 7.41(s, 1H), 7.22(d, 1H), 6.77(d, |
| | | | 1H), 6.47(s, 1H), 4.24-4.19(m, 4H), 2.28(s, 3H) |
| 12 | 252.27 | 253 | 13.27(br. s, 1H), 11.15(s, 1H), 8.98(dd, 1H), 8.78(dd, 1H), |
| | | | 8.44(dd, 1H), 7.70-7.60(m, 3H), 6.60(s, 1H), 2.34(s, 3H) |
| 13 | 226.23 | 227 | 13.33(s, 1H), 10.99(s, 1H), 9.73(s, 1H), 8.00(s, 1H), 7.87(s, |
| | | | 1H), 7.42(dd, 1H), 7.36-7.30(m, 2H), 6.76(s, 1H), 6.39(s, |
| | | | 1H) |
| 14 | 252.27 | 253 | 13.45(s, 1H), 10.31(s, 1H), 8.49(s, 1H), 8.16(d, 1H), |
| | | | 8.06(dd, 1H), 7.92(s, 1H), 7.87(d, 1H), 7.38(d, 1H), 6.82(s, 1H), |
| | | | 2.63(s, 3H) |
| 15 | 238.24 | 239 | 13.47(s, 1H), 10.46(s, 1H), 8.38(d, 1H), 8.00(t, 2H), 7.94(s, |
| | | | 1H), 7.81-7.77(m, 2H), 7.66(t, 1H), 6.84(s, 1H) |
| 16 | 245.23 | 246 | 13.01(s, 1H), 9.76(s, 1H), 7.41(s, 1H), 7.22(d, 1H), 6.77(d, |
| | | | 1H), 6.47(s, 1H), 4.24-4.19(m, 4H), 2.28(s, 3H) |
| 17 | 227.22 | 228 | 13.38(s, 1H), 12.97(s, 1H), 10.01(s, 1H), 8.24(s, 1H), |
| | | | 8.04(s, 1H), 7.89(s, 1H), 7.68(d, 1H), 7.49(d, 1H), 6.78(s, 1H) |
| 18 | 276.34 | 277 | 13.70(s, 1H), 13.42(s, 1H), 10.23(s, 1H), 8.18(s, 1H), |
| | | | 7.86(s, 1H), 7.76(dd, 1H), 7.27(d, 1H), 6.81(s, 1H) |
| 19 | 258.30 | 259 | 8.55(s, 1H), 7.98(d, 1H), 7.87(s, 1H), 7.82(dd, 1H), 7.00(s, |
| | | | 1H), 2.94(s, 3H) |
| 20 | 245.26 | 246 | 10.12(s, 1H), 8.45(d, 1H), 7.99(s, 1H), 7.81-7.73(m, 2H), |
| | | | 6.88(s, 1H) |
| 21 | 252.27 | 253 | 13.15(s, 1H), 10.48(s, 1H), 9.18(s, 1H), 8.83(s, 1H), 7.92(t, |
| | | | 1H), 7.66-7.53(m, 2H), 6.55(s, 1H), 2.30(s, 3H) |
| 22 | 238.24 | 239 | 13.60(s, 1H), 10.35(s, 1H), 8.87(d, 1H), 8.12-8.10(m, 2H), |
| | | | 8.04(d, 1H), 7.99(s, 1H), 7.81(dt, 1H), 7.67(dt, 1H) 6.89(s, |
| | | | 1H) |
| 23 | 238.24 | 239 | 13.50(s, 1H), 10.33(s, 1H), 9.25(s, 1H), 8.65(s, 1H), |
| | | | 8.20(d, 1H), 7.97-7.92(m, 2H), 7.83(dt, 1H), 7.73(dt, 1H), |
| | | | 6.87(s, 1H) |
| 24 | 268.27 | 269 | 13.49(s, 1H), 10.46(s, 1H), 9.13(s, 1H), 8.74(s, 1H), 7.94(s, |
| | | | 1H), 7.84(d, 1H), 7.36(s, 1H), 7.24(s, 1H), 6.82(s, 1H), |
| | | | 3.91(s, 3H) |
| 25 | 252.27 | 253 | 13.58(s, 1H), 10.25(s, 1H), 8.04-7.98(m, 3H), 7.94(d, 1H), |
| | | | 7.74(t, 1H), 7.58(t, 1H), 6.88(s, 1H), 2.66(s, 3H) |
| 26 | 268.27 | 269 | 13.50(s, 1H), 10.57(s, 1H), 9.21(s, 1H), 9.10(s, 1H), 7.94(s, |
| | | | 1H), 7.59-5.52(m, 2H), 7.04(d, 1H), 6.83(s, 1H), 4.00(s, |
| | | | 3H) |
| 27 | 242.28 | 243 | 13.40(1H, s), 10.11(1H, s), 8.63(1H, s), 7.87(2H, br. s), |
| | | | 6.73(1H, br. s), 2.72(4H, q), 1.81-1.69(4H, m) |
| 28 | 228.25 | 229 | 13.5(1H, br. s), 10.2(1H, br. s), 8.64(1H, d), 8.04(1H, d), |
| | | | 7.87(1H, br. s), 6.80(1H, br. s), 2.92-2.82(4H, m), 2.07(2H, |
| | | | m) |
| 29 | 292.31 | 293 | 13.46(1H, s), 10.5-10.2(1H, br. s), 8.78(1H, d), 8.04(1H, d), |
| | | | 7.86(1H, br. s), 6.80(1H, br. s), 4.56(2H, s), 3.52(2H, t), |
| | | | 3.33(2H, t) |
| 30 | 254.29 | 255 | 13.4(1H, br. s), 10.2(1H, br. s), 8.49(1H, d), 7.95(1H, d), |
| | | | 7.86(1H, br. s), 6.79(1H, br. s), 3.45-3.20(2H, m), |
| | | | 1.92-1.98(2H, m), 1.72-1.78(1H, m), 1.57-1.52(1H, m), 1.16-1.12(2H, |
| | | | m) |
| 31 | 258.28 | 259 | 13.5(1H, br. s), 10.14(1H, br. s), 8.67(1H, d), 7.85-7.94(2H, |
| | | | m), 6.79(1H, br. s), 4.84(1H, d), 4.03-3.96(1H, m), |
| | | | 2.97-2.62(4H, m), 2.00-1.85(1H, m), 1.88-1.73(1H, m) |
| 32 | 257.29 | 258 | 13.5(1H, br. s), 10.2(1H, br. s), 8.71(1H, d), 7.90(1H, d) |
| | | | 7.89(1H, br. s), 6.79(1H, br. s), 3.50(2H, s), 2.85(2H, t), |
| | | | 2.68(2H, t), 2.36(3H, s) |
| 33 | 244.25 | 245 | 13.44(1H, s), 10.26(1H, br. s), 8.72(1H, d), 7.92(1H, d), |
| | | | 7.86(1H, br. s), 6.80(1H, br. s), 4.69(2H, s), 3.96(2H, t), |
| | | | 2.83(2H, t) |
| 34 | 298.38 | 299 | 13.45(1H, br. s), 10.13(1H, br. s), 8.63(1H, d), 7.91(1H, d), |
| | | | 7.86(1H, br. s), 6.78(1H, br. s), 2.93-2.50(4H, m), |
| | | | 2.06-1.94(1H, m), 1.52-1.27(2H, m), 0.95(9H, s) |
| 35 | 343.38 | 344 | 13.51(1H, br. s), 10.32(1H, br. s), 8.71(1H, d), 8.00(1H, d), |
| | | | 7.86(1H, br. s), 6.79(1H, br. s), 4.51(2H, s), 3.64(2H, t), |
| | | | 2.81(2H, t), 1.42(9H, s) |
| 36 | 239.23 | 240 | 13.54(1H, s), 10.98(1H, s), 9.07(1H, d), 9.00(1H, d), |
| | | | 8.80(1H, dd), 7.99(1H, d), 7.88(1H, t), 7.79(1H, dd), 6.87(1H, |
| | | | d) |
| 37 | 227.22 | 228 | 13.6-12.1(2H, br. s), 10.0(1H, s), 8.16(1H, d), 8.14(1H, d), |
| | | | 7.83(1H, s), 7.51(2H, m), 6.81(1H, s) |
| 38 | 238.24 | 239 | 13.44(1H, s), 10.50(1H, s), 9.30(1H, s), 8.49(1H, d), |
| | | | 7.93(1H, d), 7.9-7.7(4H, m), 6.82(1H, s) |
| 39 | 285.30 | 286 | 13.49(1H, s), 10.28(1H, s), 8.74(1H, s), 8.09(1H, s), |
| | | | 8.01(1H, s), 7.90(1H, s), 6.78(1H, s), 4.68(0.8H, s), 4.61(1.2H, |
| | | | s), 3.75(2H, t), 2.91(1.2H, t), 2.79(0.8H, t), 2.11(3H, s)¹ |
| 40 | 272.69 | 273 | 10.4(br. s, 1H), 8.75(d, 1H), 8.05(d, 1H), 7.95-7.85(m, 2H), |
| | | | 7.80(s, 1H), 7.60-7.50(m, 1H), 6.78(s, 1H) |

¹The compound was a mixture (2:3) of the cis and trans amide forms

### Examples 41-52

### General Procedure

TBTU (0.48 mmol) was added to a solution of the relevant starting material (i.e. 3-pyrazole carboxylic acid or (iii)-(v) above; 0.40 mmol), the relevant bicyclic arylamine (0.48 mmol) and diisopropyl ethyl amine (0.80 mmol) in dry DMF (3 mL) under argon. The reaction mixture was stirred at the indicated temperature for the indicated period of time. After cooling to rt water (10 mL) was added and the mixture extracted with EtOAc (3×10 mL). The combined organic phases were washed with NaCl (aq, sat), dried (Na₂SO₄) and concentrated. The residue was purified by chromatography with EtOAc:heptane as an eluent.

TABLE 3
Examples (Ex.) 41 to 52

| | | Prepared from | | Reaction | | |
|---|---|---|---|---|---|---|
| | | (intermediate | | conditions | | |
| | | (i), (iii)-(v) | | | Temp. | |
| Ex. | Chemical name | and arylamine) | | Time h | ° C. | Yield % |
| 41 | Pyrazole-3-carboxylic acid | i | 3-Amino-6- | 18 | 80 | 10 |
| | (6-chloro- | | chloro-2- | | | |
| | 2-methylquinolin-3-yl)amide | | methylquinoline | | | |
| 42 | Pyrazole-3-carboxylic acid | i | 3-Amino-2- | 18 | 80 | 11 |
| | (2-methylquinolin-3- | | methylquinoline | | | |
| | yl)amide | | | | | |
| 43 | Pyrazole-3-carboxylic acid | i | 3-Amino-6,8- | 18 | 80 | 10 |
| | (6,8-dibromo-2- | | dibromo-2- | | | |
| | methylquinolin-3-yl)amide | | methylquinoline | | | |
| 44 | 4-Bromopyrazole-3- | iv | 3-Amino- | 18 | 20 | 16 |
| | carboxylic acid quinolin-3- | | quinoline | | | |
| | ylamide | | | | | |
| 45 | 5-Trifluoromethyl- | v | 3-Amino- | 18 | 20 | 4 |
| | pyrazole-3-carboxylic acid | | quinoline | | | |
| | quinolin-3-ylamide | | | | | |
| 46 | Pyrazole-3-carboxylic acid | i | 3-Amino-6,7,8,9- | 2 | 60 | 34 |
| | (6,7,8,9-tetrahydro-5H- | | tetrahydro-5H- | | | |
| | cyclohepta[b]pyrid-3- | | cyclohepta[b]- | | | |
| | yl)amide | | pyridine | | | |
| 47 | 5-Chloropyrazole-3- | iii | 3-Amino- | 2 | 60 | 6 |
| | carboxylic acid quinolin-3- | | quinoline | | | |
| | ylamide | | | | | |
| 48 | 5-Chloropyrazole-3- | iii | 4-Aminoindole | 3 | 60 | 29 |
| | carboxylic acid indol-4- | | | | | |
| | ylamide | | | | | |
| 49 | Pyrazole-3-carboxylic acid | i | 3-Amino-6,7- | 18 | 80 | 30 |
| | (6,7-dimethoxyquinolin-3- | | dimethoxy- | | | |
| | yl)amide | | quinoline | | | |
| 50 | 5-Chloropyrazole-3- | iii | 4-Amino- | 5 | 60 | 4 |
| | carboxylic acid quinolin-4- | | quinoline | | | |
| | ylamide | | | | | |
| 51 | Pyrazole-3-carboxylic acid | i | 8-Amino-(2,3- | 18 | 80 | 4 |
| | (2,3-dihydro- | | dihydro- | | | |
| | [1,4]dioxino[2,3- | | [1,4]dioxano[2,3- | | | |
| | g]quinolin-8-yl)amide | | g]quinoline | | | |
| 52 | Pyrazole-3-carboxylic acid | i | 3-Amino-6,7- | 18 | 80 | 3 |
| | (6,7-methylenedioxy | | methylenedioxy- | | | |
| | quinolin-3-yl)amide | | quinoline | | | |

TABLE 4
Physical properties of the compounds of Examples 41-52

| | | MS | |
|---|---|---|---|
| | | (M⁺+1), | |
| Ex. | M.W. | m/z | ¹H NMR(DMSO-d₆, 400MHz), δ |
| 41 | 286.72 | 287 | 13.64(s, 1H), 9.88(s, 1H), 8.55(s, 1H), 8.10(s, 1H), |
| | | | 7.95-7.93(m, 2H), 7.68(dd, 1H), 6.85(s, 1H), 2.68(s, 3H) |
| 42 | 252.27 | 253 | 13.49(s, 1H), 9.82(s, 1H), 8.52(s, 1H), 7.95(s, 1H), 7.93(d, |
| | | | 2H), 7.68(t, 1H), 7.54(t, 1H), 6.82(s, 1H), 2.67(s, 3H) |
| 43 | 410.06 | 411 | 13.54(s, 1H), 9.88(s, 1H), 8.64(s, 1H), 8.31(s, 1H), 8.19(s, |
| | | | 1H), 7.96(s, 1H), 6.84(s, 1H), 2.73(s, 3H) |
| 44 | 317.14 | 317 | 13.9(br. s, 1H), 10.67(s, 1H), 9.16(d, 1H), 8.87(d, 1H), |
| | | 319 | 8.21(s, 1H), 7.96(t, 2H), 7.66(t, 1H), 7.59(t, 1H) |
| 45 | 306.24 | 307 | 14.76(s, 1H), 10.81(s, 1H), 9.12(d, 1H), 8.78(d, 1H), |
| | | | 8.00(d, 2H), 7.71(dt, 1H), 7.64-7.59(m, 2H) |
| 46 | 256.30 | 257 | 13.43(1H, s), 10.11(1H, s), 8.62(1H, s), 7.95-7.85(2H, m), |
| | | | 7.90(1H, s), 6.77(1H, br. s), 2.95-2.90(2H, m), |
| | | | 2.75-2.70(2H, m), 1.88-1.75(2H, m), 1.68-1.45(4H, m) |
| 47 | 272.69 | 273 | 14.13(br. s, 1H), 10.68(s, 1H), 9.11(d, 1H), 8.76(d, 1H), |
| | | | 8.00(d, 1H), 7.98(d, 1H), 7.70(dt, 1H), 7.61(dt, 1H), 7.16(s, 1H) |
| 48 | 260.68 | 261 | 13.96(br. s, 1H), 11.18(br. s, 1H), 10.10(br. s, 1H), |
| | | | 7.34-7.21(m, 4H), 7.08(t, 1H), 6.54(s, 1H) |
| 49 | 298.30 | 299 | 13.49(s, 1H), 10.40(s, 1H), 8.95(s, 1H), 8.65(s, 1H), 7.91(s, |
| | | | 1H), 7.30-7.27(m, 2H), 6.81(s, 1H), 3.90(s, 6H) |
| 50 | 272.69 | 273 | 14.14(br. s, 1H), 10.65(br. s, 1H), 8.89(d, 1H), 8.22(d, 1H), |
| | | | 8.04(d, 1H), 7.87(d, 1H), 7.82(dt, 1H), 7.65(dt, 1H), 7.33(s, |
| | | | 1H) |
| 51 | 296.28 | 297 | 13.4(s, 1H), 10.04(s, 1H), 8.94(s, 1H), 8.60(s, 1H), 7.91(s, |
| | | | 1H), 7.32(s, 2H), 6.80(s, 1H), 4.35(s, 4H) |
| 52 | 282.25 | 283 | 8.94(s, 1H), 8.62(s, 1H), 7.77-7.71(m, 1H), 7.57-7.50(m, |
| | | | 1H), 7.33-7.21(m, 2H), 6.83(s, 2H) |

### Examples 53-55

### General Procedure

Lithium diisopropylamide (1.6 M in THF, 0.78 mL, 1.25 mmol) was added dropwise to a stirred solution of the relevant bicyclic arylamine (0.50 mmol) in 1,4-dioxane (15 mL) and the mixture was stirred at rt for 1 h. Dipyrazolo[1,5a;1′,5′-d]pyrazine-4,9-dione (intermediate (i)) was added in one portion and the mixture was stirred at the indicated temperature for the indicated period of time. After cooling to rt the mixture was concentrated in vacuo and water (10 mL) was added. The mixture was extracted with EtOAc (3×15 mL), and the combined organic phases were dried (MgSO₄) and concentrated. The title compounds were obtained after chromatography using EtOAc:hexane (3:2) as eluent.

TABLE 5
Examples (Ex.) 53 to 55

| | | Prepared from | | Reaction | | |
|---|---|---|---|---|---|---|
| | | (intermediate (i) | | conditions | | |
| Ex. | Chemical name | and arylamine) | | Time h | Temp. ° C. | Yield % |
| 53 | Pyrazole-3-carboxylic acid | i | 2-Amino-3- | 18 | 80 | 17 |
| | (3-cyano-6-methyl-4,5,6,7- | | cyano-6-methyl- | | | |
| | tetrahydrobenzo-[b]thien-2- | | 4,5,6,7- | | | |
| | yl)amide | | tetrahydrobenzo- | | | |
| | | | [b]thiophene | | | |
| 54 | 2-[(Pyrazole-3- | i | 2-Amino-4,7- | 18 | 40 | 35 |
| | carbonyl)amino]-4,7- | | dihydro-5H- | | | |
| | dihydro-5H-thieno[2,3- | | thieno[2,3- | | | |
| | c]pyridine-3,6- | | c]pyridine-3,6- | | | |
| | dicarboxylic acid 6-tert-butyl | | dicarboxylic acid | | | |
| | ester 3-ethyl ester | | 6-tert-butyl ester | | | |
| | | | 3-ethyl ester | | | |
| 55 | Pyrazole-3-carboxylic acid | i | 2-Amino-3- | 3 | 40 | 39 |
| | (3-cyano-5,6,7,8- | | cyano-5,6,7,8- | | | |
| | tetrahydro-4H- | | tetrahydro-4H- | | | |
| | cyclohepta[b]thien-2-yl)amide | | cyclohepta- | | | |
| | | | [b]thiophene | | | |

TABLE 6
Physical properties of the compounds of Examples 53-55

| | | MS | |
|---|---|---|---|
| | | (M⁺+1), | |
| Ex. | M.W. | m/z | ¹H NMR(DMSO-d₆, 400MHz), δ |
| 53 | 286.35 | 287 | 13.62(s, 1H), 11.00(s, 1H), |
| | | | 7.96(d, 1H), 7.94(d, 1H), |
| | | | 6.85(d, 1H), 6.83(d, 1H), 2.74(dd, 1H), |
| | | | 2.60-2.50(m, 2H), |
| | | | 1.83(dd, 1H), 1.90-1.81(m, 2H), 1.44-1.33(m, |
| | | | 1H), 1.03(d, 3H) |
| 54 | 420.48 | 421 | 13.64(br. s, 1H), 12.21(br s, 1H), |
| | | | 7.98(d, 1H), 6.85(d, 1H), |
| | | | 4.47(br. s, 2H), 4.30(q, 2H), 3.56(t, |
| | | | 2H) 2.97(t, 2H), 1.42(s, |
| | | | 9H), 1.34(t, 3H) |
| 55 | 286.35 | 287 | 16.62(br. s, 1H), 10.91(br. s, 1H), |
| | | | 7.96(s, 1H), 6.83(d, 1H), |
| | | | 2.66-2.74(m, 4H), 1.82(br. s, 2H), |
| | | | 1.65-1.55(m, 4H) |

### Example 56

### 2-[(Pyrazole-3-carbonyl)amino]-4,5,6,7-tetrahydrobenzo[b]thiophene-3-carboxylic acid ethyl ester

Lithium diisopropylamide (1.8 M in heptane:THF:ethylbenzene, 0.32 mL, 0.5 mmol) was added dropwise to a stirred solution of 2-amino-4,5,6,7-tetrahydro-benzo[b]thiophenecarboxylic acid ethyl ester (113 mg, 0.5 mmol) in THF (25 mL) at −78° C. The mixture was allowed to warm to rt and recooled to −78° C. Dipyrazolo[1,5-a;1′,5′-d]pyrazine-4,9-dione (intermediate (i)) (94 mg, 0.5 mmol) was added in one portion and the mixture allowed to warm to rt and stirred for 18 h. The yellow precipitate was collected, washed with water (25 mL) and isohexane (50 mL) and recrystallised from DMF (3 mL) to give the title compound (109 mg, 68%) as a pale yellow solid.

¹H NMR (DMSO-d₆, 400 MHz) δ 12.23 (s, 1H), 7.98 (s, 1H), 6.84 (s, 1H), 4.31 (q, 2N), 2.74 (br. s; 2H), 2.63 (br. s, 2H), 1.74 (br. s, 4H), 1.35 (t, 3H).

¹³C NMR (DMSO-d₆, 100 MHz) δ 165.7, 159.0, 146.6, 145.0, 132.0, 131.2, 126.6, 111.8, 106.5, 60.9, 26.4, 24.3, 23.0, 22.9, 14.7.

### Example 57

### Pyrazole-3-carboxylic acid (3,4-(difluoromethylenedioxy)phenyl)amide

A mixture of 3,4-(difluoromethylenedioxy)aniline (346 mg, 2.0 mmol) and dipyrazolo[1,5-a;1′,5′-d]pyrazine-4,9-dione (intermediate (i)) in DMF (5 mL) was stirred at 120° C. for 18 h. The solvent was removed in vacuo and water (10 mL) was added to the residue. The mixture was extracted with EtOAc (3×10 mL), and the combined extracts were filtered through silica gel and concentrated. The title compound was precipitated by addition of isohexane and recrystallised from toluene (10 mL). Yield: 241 mg (45%) as a white solid.

MS (M⁺+H) m/z=268.

¹H NMR (DMSO-d₆, 400 MHz) δ 10.35 (s, 1H), 7.95 (s, 1H), 7.92 (s, 1H), 7.63 (d, 1H), 7.37 (d, 1H), 6.79 (s, 1H).

¹³C NMR (DMSO-d₆, 100 MHz) δ 161.2, 146.9, 143.0, 138.9, 136.2, 131.9 (t, J=252 Hz), 131.1 116.3, 110.4, 106.4, 103.4.

### Example 58

### Pyrazole-3-carboxylic acid benzothiazol-6-ylamide

A mixture of dipyrazolo[1,5-a;1′,5′-d]pyrazine-4,9-dione (intermediate (i)) (100 mg, 0.53 mmol), benzothiazol-6-ylamine (239 mg, 1.59 mmol), DMAP (65 mg, 0.53 mmol) and DMF (5 mL) was stirred at 140° C. for 18 h. After cooling to rt, the mixture was poured into water and extracted with EtOAc (3×10 mL). The combined extracts were dried (Na₂SO₄) and concentrated. Purification by chromatography (EtOAc:isohexane 9:1) and recrystallisation from MeOH:water afforded the title compound (42 mg, 16%) as white crystals.

MS (M⁺+H) m/z=245.

¹H NMR (CD₃OD, 400 MHz) δ 9.15 (s, 1H), 8.68-8.62 (m, 1H), 8.06-7.98 (m, 1H), 7.84-7.72 (m, 2H), 6.94-6.85 (m, 1H).

### Example 59

### Pyrazole-3-carboxylic acid quinolin-8-ylamide

A mixture of dipyrazolo[1,5-a;1′,5′-d]pyrazine-4,9-dione (intermediate (i)) (522 mg, 2.77 mmol), 8-aminoquinoline (200 mg, 1.39 mmol), DMAP (169 mg, 1.39 mmol) and DMF (5 mL) was stirred at 140° C. for 2 h. The mixture was cooled to rt and concentrated. Purification by chromatography (diethyl ether) and recrystallisation from MeOH:water afforded the title compound (213 mg, 64%) as white crystals.

MS (M⁺+H) m/z=239.

¹H NMR (CD₃OD, 400 MHz) δ 13.5 (br. s, 1H), 11.2 (s, 1H), 9.00-8.89 (m, 1H), 8.83-8.70 (m, 1H), 8.48-8.35 (m, 1H), 8.00-7.90 (m, 1H), 7.73-7.55 (m, 3H), 6.90-6.80 (m, 1H).

### Example 60

### Pyrazole-3-carboxylic acid (2,3-(difluoromethylenedioxy)phenyl)amide

A mixture of dipyrazolo[1,5-a;1′,5′-d]pyrazine-4,9-dione (intermediate (i)) (100 mg, 0.53 mmol), 2,3-(difluoromethylenedioxy)aniline (276 mg, 1.59 mmol), DMAP (65 mg, 0.53 mmol) and DMF (5 mL) was stirred at 140° C. for 18 h. The mixture was cooled to rt and concentrated. Purification by chromatography (EtOAc) and preparative LC-MS afforded the title compound (34 mg, 12%) as a white solid.

MS (M⁺+H) m/z=268.

¹H NMR (CD₃OD, 400 MHz) δ 10.4 (br s, 1H), 7.75-7.60 (m, 1H), 7.50-7.30 (m, 1H), 7.15-6.65 (m, 3H)

¹³C NMR (MeOH-d₄, 100 MHz) δ 160.4, 143.9, 136.6, 131.5 (t, J=252 Hz), 124.7, 122.1, 121.0, 107.1, 106.5.

### Example 61

### Pyrazole-3-carboxylic acid (indazol-6-yl)amide

A mixture of dipyrazolo[1,5-a;1′,5′-d]pyrazine-4,9-dione (intermediate (i)) (1.41 g, 7.51 mmol), 6-aminoindazole (1.06 g, 7.93 mmol), DMAP (0.46 g, 3.76 mmol) and chloroform (15 mL) was stirred at 80° C. for 18 h. Concentration and purification by chromatography (diethyl ether) followed by recrystallisation from EtOAc:isohexane afforded the title compound (75 mg, 9%) as white crystals.

MS (M⁺+H) m/z=228.

¹H NMR (CD₃OD, 400 MHz) δ 8.38-7.95 (m, 2H), 7.90-7.43 (m, 4H), 7.05-6.80 (m, 1H).

### Example 62

### 4-Methylpyrazole-3-carboxylic acid quinolin-8-ylamide

### (a) 4-Methyl-5-trimethylsilylpyrazole-3-carboxylic acid ethyl ester

A mixture of 1-trimethylsilyl-1-propyne (1.0 g, 8.93 nmol) and ethyl diazoacetate (1.0 g, 8.77 mmol) was stirred at 80° C. for 1 day and then at 100° C. for 2 days. The mixture was diluted with 50% aq. EtOH (10 mL) and the precipitate was collected and dried to give 478 mg (24%) of the sub-title compound as a pale yellow solid.

¹H NMR (CDCl₃, 400 MHz) δ 10.36 (br. s, 1H), 4.30 (q, 2H), 2.34 (s, 3H), 1.25 (t, 3H), 0.30 (s, 9H).

¹³C NMR (CDCl₃, 100 MHz) δ 163.7, 143.3, 141.2, 127.7, 60.9, 14.3, 10.5, −1.5.

### (b) 4-Methyl-5-trimethylsilylpyrazole-3-carboxylic acid

NaOH (aq., 1 M, 73 mL, 73 mmol) was added to a solution of 4-methyl-5-trimethylsilylpyrazole-3-carboxylic acid ethyl ester (3.3 g, 14.6 mmol) in EtOH (100 mL). The mixture was heated to 80° C. for 15 min, cooled to rt, acidified with hydrochloric acid and concentrated to near dryness. The residue was extracted with EtOAc (3×100 mL) and the combined extracts dried (NaSO₄) and concentrated to give 2.50 g (86%) of the sub-title compound as a pale yellow solid.

¹H NMR (DMSO-d₆, 400 MHz) δ 11.80 (br. s, 1H), 2.27 (s, 3H), 0.30 (s, 9H).

¹³C NMR (DMSO-d₆, 100 MHz) δ 164.8, 141.8, 140.7, 126.4, 10.8, ˜0.5.

### (c) 3,8-Dimethyl-2,7-bis-trimethylsilyldipyrazolo[1,5-a;1′,5′-d]pyrazine-4,9-dione

A mixture of 4-methyl-5-trimethylsilylpyrazole-3-carboxylic acid (50 mg, 0.25 mmol), EDCI (73 mg, 0.38 mmol), DMAP (46 mg, 0.38 mmol) and dry CH₂Cl₂ (4 mL) was stirred at 50° C. for 2 days. The solution was diluted with CH₂Cl₂ (20 mL), washed with water (2×5 mL), filtered through silica gel and concentrated. Addition of isohexane gave the sub-title compound (31 mg, 75%) as a white solid.

MS (M⁺+H) m/z 361.

¹H NMR (CDCl₃, 400 MHz) δ 2.57 (s, 6H), 0.41 (s, 18H).

¹³C NMR (CDCl₃, 100 MHz) δ 162.9, 149.5, 136.9, 130.4, 10.7, −1.3.

### (d) 4-Methyl-5-trimethylsilylpyrazole-3-carboxylic acid quinolin-8-ylamide

A mixture of 3,8-dimethyl-2,7-bis-trimethylsilyldipyrazolo[1,5-a;1′,5′-d]pyrazine-4,9-dione (500 mg, 1.39 mmol), 8-aminoquinoline (1.0 g, 6.95 mmol) and DMAP (340 mg, 2.78 mmol) was heated under argon at 120° C. for 2½ h and then cooled to rt. EtOH (5 mL), water (15 mL) and n-heptane (15 mL) were added and the precipitation was filtered off and purified by chromatography to give the sub-title compound (220 mg, 25%).

¹H NMR (DMSO-d₆, 400 MHz) δ 13.07 (br. s, 1H), 11.28 (s, 1H), 8.95 (dd, 1H), 8.79 (dd, 1H), 8.44 (dd, 1H), 7.68-7.60 (m, 3H), 2.43 (s, 3H), 0.35 (s, 9H).

### (e) 4-Methylpyrazole-3-carboxylic acid quinolin-8-ylamide

A mixture of 4-methyl-5-trimethylsilylpyrazole-3-carboxylic acid quinolin-8-ylamide (220 mg, 0.67 mmol), TBAF (175 mg, 0.67 mmol) and dry THF (10 mL) was stirred at rt for 24 h under argon. Water (10 mL) was added and the precipitate which formed was filtered off. The organic layer of the filtrate was concentrated and purified by chromatography. The fraction of interest was pooled with the initial precipitate to give the title compound (130 mg, 75%).

¹H NMR (DMSO-d₆, 400 MHz) δ 13.22 (br. s, 1H), 11.22 (s, 1H), 8.97 (dd, 1H), 8.80 (dd, 1H), 8.44 (dd, 1H), 7.78 (s, 1H), 7.69-7.60 (m, 3H), 2.34 (s, 3H).

### Example 63

### Pyrazole-3-carboxylic acid (6-bromoquinolin-4-yl)amide

A mixture of dipyrazolo[1,5-a;1′,5′-d]pyrazine-4,9-dione (intermediate (i)) (94 mg, 0.50 mmol), 4-amino-6-bromoquinoline (112 mg, 0.50 mmol), DMAP (61 mg, 0.50 mmol) and DMF (5 mL) was stirred at 120° C. for 42 h. The solution was cooled to rt and the precipitate formed was filtered and washed with MeOH:water (4:1). Crystallisation from EtOH:water gave the title product (35 mg, 22%) as a white powder.

MS (M⁺+H) m/z 317.

¹H NMR (DMSO-d₆, 400 MHz) δ 13.61 (br. s, 1H), 10.55 (br. s, 1H), 8.90 (d, 1H), 8.32 (s, 1H), 7.90-8.06 (m, 4H), 6.69 (s, 1H).

### Example 64

### Pyrazole-3-carboxylic acid (6-trifluoromethylquinolin-4-yl)amide

A mixture of dipyrazolo[1,5-a;1′,5′-d]pyrazine-4,9-dione (intermediate (i)) (94 mg, 0.50 mmol), 4-amino-6-trifluoromethylquinoline (106 mg, 0.50 mmol), DMAP (61 mg, 0.50 mmol) and DMF (5 mL) was stirred at 120° C. for 42 h. The solution was cooled to rt, water (5 mL) was added and the mixture was extracted with EtOAc (3×5 mL). The combined organic phases were washed with water (10 mL) and NaCl (aq, sat). The solvent was removed in vacuo and the residue crystallised from EtOAc:pentane to give the title product (101 mg, 66%) as a white powder.

MS (M⁺+H) m/z 307.

¹H NMR (DMSO-d₆, 400 MHz) δ 13.63 (br. s, 1H), 10.78 (br. s, 1H), 9.01 (d, 1H), 8.55 (s, 1H), 8.21 (d, 1H), 8.14 (d, 1H), 7.99-8.04 (m, 2H), 6.90 (s, 1H).

### Example 65

### 4-Methylpyrazole-3-carboxylic acid quinolin-3-ylamide

### (a) 4-Methylpyrazole-3-carboxylic acid

4-Methyl-5-trimethylsilylpyrazole-3-carboxylic acid ethyl ester (3.3 g, 14.6 mmol), prepared in accordance with Example 62(a), was dissolved in EtOH (100 mL) and NaOH (aq., 1 M, 73 mL, 73 mmol) was added. The mixture was heated at reflux for 18 h, cooled to rt, acidified with hydrochloric acid (1 M, 100 mL) and concentrated to near dryness. The residue was extracted with EtOAc (3×100 mL), the combined extracts dried with (Na₂SO₄) and concentrated to give the sub-title compound (1.90 g (99%)).

¹H NMR(DMSO-d₆, 400 MHz) δ 13 (br. s, 2H), 7.50 (s, 1H), 2.19 (s, 3H).

### (b) 4-Methylpyrazole-3-carboxylic acid quinolin-3-ylamide

A mixture of 4-methylpyrazole-3-carboxylic acid (100 mg, 0.80 mmol), 3-aminoquinoline (137 mg, 0.95 mmol), diisopropylamine (161 mg, 1.59 mmol), HATU (361 mg, 0.95 mmol) and DMF (10 mL) was stirred at rt for 18 h and heated to 50° C. for 4 h. Diethyl ether (25 mL) was added, the layers separated and the organic phase washed with CaCl₂ (aq, sat) and concentrated. Purification by chromatography (EtOAc:hexane) gave the title compound (70 mg, 30%) as a grey powder.

MS (M⁺+H) m/z 253.

¹H NMR (DMSO-d₆, 400 MHz) δ 13.21 (s, 1H), 10.44 (s, 1H), 9.17 (s, 1H), 8.87 (s, 1H), 7.92 (t, 1H), 7.71 (s, 1H) 7.65-7.53 (m, 2H), 2.29 (s, 3H)

### Example 66

### Pyrazole-3-carboxylic acid (5,6,7,8-tetrahydro[1,6]naphthyridin-3-yl)amide

A solution of 3-[(pyrazole-3-carbonyl)amino]-7,8-dihydro-5H-[1,6]naphthyridine-6-carboxylic acid tert-butyl ester (82 mg, 0.239 mmol, see Example 35) in trifluoroacetic acid: CH₂Cl₂ (1:5, 5 mL) was stirred at rt for 45 min and concentrated. The residue was treated with EtOAc (20 mL) and a mixture of Na₂CO₃ (2 M, 10 mL), NaOH (4 M, 1 mL) and NaCl (aq, sat, 5 mL). The layers were separated and the aqueous phase extracted with EtOAc (4×5 mL). The combined organic phases were dried (Na₂SO₄) and concentrated. The residue was crystallised from MeOH to give the title compound (13 mg, 22%) as a white solid.

MS (M⁺+H) m/z 244.

¹H NMR (DMSO-d₆, 400 MHz) δ 13.47 (br. s, 1H), 10.17 (s, 1H), 8.66 (d, 1H), 7.88-7.84 (m, 2H), 6.79 (d, 1H), 3.83 (s, 2H), 3.00 (t, 2H), 2.71 (t, 2H).

### Example 67

### Pyrazole-3-carboxylic acid (6-hydroxyquinolin-3-yl)amide

A mixture of pyrazole-3-carboxylic acid (6-hydroxy-5,6,7,8-tetrahydroquinolin-3)-yl)amide (66 mg, 0.26 nmol; see Example 31), mesitylene (4 mL) and palladium on active carbon (10% Pd, 90 mg) was heated under argon in a sealed vial at 200° C. for 1 h. After cooling to rt, MeOH (20 mL) was added and the mixture was filtered through Celite®. The filtrate was concentrated and the residue crystallised from MeOH to give the title product (13 mg, 21%) as an off-white solid.

MS (M⁺+H) m/z 255.

¹H NMR (DMSO-d₆, 400 MHz) δ 13.47 (s, 1H), 10.43 (s, 1H), 9.95 (s, 1H), 9.95 (d, 1H), 8.58 (d, 1H), 7.92 (d, 1H), 7.77 (d, 1H), 7.17 (dd, 1H), 7.05 (d, 1H), 6.80 (d, 1H).

### Example 68

### 4-Fluoropyrazole-3-carboxylic acid quinolin-3-ylamide

### (a) 4-Fluoropyrazole-3-carboxylic acid ethyl ester

The sub-title compound was prepared from pyrazole-3-carboxylic acid ethyl ester in accordance with a literature procedure (R. Storer, et al., Nucleosides & Nucleotides 18, 203 (1999). A mixture (˜2:1) of sub-title compound and unreacted starting material was obtained and used without further purification.

### (b) 4-Fluoropyrazole-3-carboxylic acid

Aqueous NaOH (2M, 18 mmol, 9 mL) was added to a solution of a mixture (˜2:1) of 4-fluoropyrazole-3-carboxylic acid ethyl ester and pyrazole-3-carboxylic acid ethyl ester (1.2 g, ˜8 mmol; see step (a) above) in dioxane (9 mL) at room temperature and was stirred for 16 h. A second portion of aqueous NaOH (2M, 18 mmol, 9 mL) was added and the mixture was stirred for another 4 h. The mixture was acidified with aqueous HCl (2M, 20 mL), concentrated, stirred with MeOH (30 mL) and filtered. The filtrate was concentrated and the residue was crystallised with aqueous HCl (0.01M) to give a mixture (˜3:1) of the sub-title compound and pyrazole-3-carboxylic acid as a white solid (Yield: 267 mg (˜2 mmol, ˜25%)). This mixture was employed without further purification.

¹H-NMR (DMSO-d₆): δ 13.7-13.1 (br s, 1.3H), 7.9-7.7 (m, 1H), 7.73 (d, 0.3H), 6.70 (d, 0.3H).

### (c) 4-Fluoropyrazole-3-carboxylic acid quinolin-3-ylamide

TBTU (242 mg, 0.75 mmol) was added to a solution of a mixture (˜3:1) of 4-fluoropyrazole-3-carboxylic acid and pyrazole-3-carboxylic acid (85 mg, 0.69 mmol; see step (b) above), 3-aminoquinoline (128 mg, 0.89 mmol) and diisopropyl ethyl amine (239 μL, 1.37 mmol) in dry DMF (2 mL). The mixture was stirred at rt for 3 days and at 85° C. for 18 h. An additional amount of TBTU (36 mg, 0.10 mmol) was added and the mixture was stirred at 85° C. for another 18 h and at rt for 5 days. Water (10 mL) was added and the mixture was extracted with EtOAc (3×10 mL). The combined organic phases were dried (Na₂SO₄) and concentrated. The residue was purified by chromatography (gradient: heptane to EtOAc:triethyl amine (49:1)) to give a mixture of the title compound and pyrazole-3-carboxylic acid quinolin-3-ylamide (˜10:1, 77 mg, ˜44%) as a yellow solid.

MS (M⁺+H) m/z 257.

¹H NMR (DMSO-d₆, 400 MHz) δ 13.50 (s, 1H), 10.59 (s, 1H), 9.15 (d, 1H), 8.80 (d, 1H), 8.09-8.03 (m, 1H), 7.94 (m, 2H), 7.62 (ddd, 1H), 7.57 (ddd, 1H).

### Example 69

### Pyrazole-3-carboxylic acid [1,6]naphthyridin-3-ylamide

### (a) 3-[(Pyrazole-3-carbonyl)amino]-5,6,7,8-tetrahydro[1,6]naphthyridin-6-ium trifluoroacetate

A solution of 3-[(pyrazole-3-carbonyl)amino]-7,8-dihydro-5H-[1,6]naphthyridine-6-carboxylic acid tert-butyl ester (270 mg, 0.786 mmol; see Example 36) and trifluoroacetic acid (5.5 mL) in CH₂Cl₂ (30 mL) was stirred at rt for 1 h and concentrated. The residue was crystallised from acetonitrile:diethyl ether to give the sub-title compound (147 mg, 52%) as a light brown solid.

¹H NMR (DMSO-d₆, 400 MHz) δ 13.54 (s, 1H), 10.39 (s, 1H), 9.25 (s, 2H), 8.79 (d, 1H), 8.14 (d, 1H), 7.88 (d, 1H), 6.80 (s, 1H), 4.34 (s, 2H), 3.48 (br. s, 2H), 3.04 (t, 2H).

### (b) Pyrazole-3-carboxylic acid[1,6]naphthyridin-3-ylamide

A mixture of 3-[(pyrazole-3-carbonyl)amino]-5,6,7,8-tetrahydro [1,6]naphthyridin-6-ium trifluoroacetate (141 mg, 0.39 mmol), pyridine (63 μL), mesitylene (3 mL) and palladium on active carbon (10% Pd, 141 mg) was heated under argon in a sealed vial at 200° C. for 50 min. After cooling to rt MeOH (20 mL) was added and the mixture was filtered through Celite®. The filtrate was concentrated and the residue purified by chromatography (EtOAc:heptane) to give the title product (8 mg, 8%) as a yellow solid.

MS (M⁺+H) m/z 240.

¹H NMR (DMSO-d₆, 400 MHz) δ 13.54 (s, 1H), 10.78 (s, 1H), 9.43 (s, 1H), 9.03 (s, 1H), 8.64 (d, 1H), 8.24 (m, 1H), 7.94 (s, 1H), 7.83 (d, 1H), 6.85 (s, 1H).

### Example 70

### 4-Methylpyrazole-3-carboxylic acid (2,3-(difluoromethylenedioxy)phenyl)amide

### (a) 4-Methylpyrazole-3-carboxylic acid

A mixture of 4-methyl-5-trimethylsilylpyrazole-3-carboxylic acid ethyl ester (Example 62(a)) (3.30 g, 14.6 mmol), NaOH (aq., 1M, 73 mL, 73 mmol) and EtOH (100 mL) was heated at reflux for 18 h. The mixture was acidified with HCl (aq, 1 M, 80 mL) and concentrated. The residue was extracted with EtOAc (3×100 mL) and the combined extracts were dried (NaSO₄) and concentrated to give the sub-title product in quantitative yield (1.90 g).

¹H NMR (DMSO-d₆, 400 MHz) δ 13.00 (broad s, 2H), 7.50 (s, 1H), 2.19 (s, 3H).

### (b) 4-Methylpyrazole-3-carboxylic acid (2,3-(difluoromethylenedioxy)phenyl)-amide

EDCI (1.71 g, 8.90 mmol) and DMAP (1.09 g, 8.90 mmol) were dissolved in dry CH₂Cl₂ (20 mL) under argon. 2,3-(Difluoromethylenedioxy)aniline (1.55 g, 8.90 mmol) followed by a solution of 4-methylpyrazole-3-carboxylic acid (750 mg, 5.95 mmol) in DMF:CH₂Cl₂ (1:1, 10 mL) were added and the mixture was heated at reflux for 18 h. The mixture was concentrated to near dryness. EtOAc (50 mL) was added and the organic phase was collected and washed with CaCl₂ (aq, sat, 2×20 mL), dried (NaSO₄) and concentrated. The product was precipitated by addition of heptane (30 mL). Yield: 500 mg (30%) of a white solid.

¹H NMR (DMSO-d₆, 400 MHz) δ 13.20 (1H, s), 9.99 (1H, s), 7.69 (1H, s), 7.38 (1H, dd), 7.21-7.16 (2H, m), 2.25 (3H, s).

### Example 71

Title compounds of the Examples were tested in the biological test described above and were found to exhibit an IC₅₀ of below 10 μM. For example, the following representative compounds of the examples exhibited the following IC₅₀ values:

Example 2: 330 nM

Example 3: 843 nM

Example 4: 72 nM

Example 5: 970 nM

Example 7: 420 nM

Example 48: 211 nM

Example 56: 76 nM

Example 59: 465 nM

Example 60: 390 nM

Example 63: 682 nM

1. A compound of formula I, 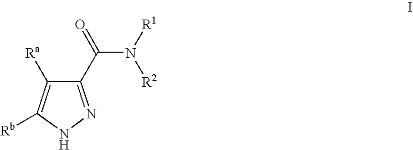
wherein
R¹ represents a bicyclic heterocyclic group, which group is optionally substituted by one or more substituents selected from B¹ and comprises:
(a) 1 to 4 heteroatoms selected from nitrogen, oxygen and sulfur;
(b) a 5- or 6-membered aromatic ring, which ring is attached to the rest of the compound of formula I via the essential —N(R²)— group of the latter; and
(c) a 5- or 6-membered aromatic, or a 4- or 8-membered non-aromatic, ring, which ring is attached to the other ring via two atoms that are common to both rings and are adjacent to each other;
R² represents H or C₁₋₆ alkyl, which latter group is optionally substituted by one or more halo groups;
B¹ represents —OH, cyano, halo, nitro, C₁₋₆ alkyl, (which latter group is optionally substituted by one or more halo atoms), —OR^{3x}, —N(R^{3a})R^{3b}, —C(O)R^{3c}, —C(O)OR^{3d}, —C(O)N(R^{3e})R^{3f}, —N(R^{3g})C(O)R^{3h}, —N(R³ⁱ)C(O)N(R^{3j})R^{3k}, —N(R^{3m})S(O)₂R^{4a}, —S(O)ₚR^{4b}, —OS(O)₂R^{4c}, —S(O)₂N(R³ⁿ)R^{3p} or, provided that it is not attached to a ring that is aromatic in its nature, ═O;
R^{a} and R^{b} independently represent H, halo, cyano, C₁₋₆ alkyl (which alkyl group is optionally substituted by one or more halo or C₁₋₆ alkoxy groups (which alkoxy group may itself be substituted by one or more halo group)), C₁₋₆ alkoxy (which alkoxy group is optionally substituted by one or more halo atoms) or —N(R^{3q})R^{3r};
R^{3a} to R^{3r} and R^{4b} independently represent H or C₁₋₆ alkyl (which alkyl group is optionally substituted by one or more halo atoms);
R^{4a}, R^{4c} and R^{3x} independently represent C₁₋₆ alkyl (which alkyl group is optionally substituted by one or more halo atoms); or
any pair of R^{3a}, R^{3b}, R^{3e} to R^{3r} and R^{4a} may, for example when present on the same or adjacent atoms, be linked together to form, with those, or other relevant, atoms, a 3- or 8-membered ring, optionally containing a further 1 to 3 heteroatoms and/or 1 to 3 unsaturations, which ring is itself optionally substituted by one or more substituents selected from halo and C₁₋₆ alkyl (which alkyl group is optionally substituted by one or more halo atoms); or
when R¹ is substituted by two —OR^{3x} groups that are adjacent to each other, these groups may be linked to form, together with the oxygen atoms to which they are attached, a 5- or 6-membered ring optionally containing 1 further heteroatom and 1 unsaturation, which ring is itself optionally substituted by one or more substituents selected from halo and C₁₋₆ alkyl (which alkyl group is optionally substituted by one or more halo atoms); and
p represents 0, 1 or 2;
or a pharmaceutically-acceptable salt thereof,
provided that, when R^{b} and R² both represent H, and;
(A) R^{a} represents H, then R¹ does not represent a benzo[1,3]dioxol-5-yl group;
(B) R^{a} represents chloro, then R¹ does not represent a benzothiazol-2-yl group;
(C) R^{a} represents iodo, then R¹ does not represent a benzothiazol-2-yl, a 6-methoxybenzothiazol-2-yl, or a 1-methyl-1H-benzoimidazol-2-yl, group;
(D) R^{a} represents bromo, then R¹ does not represent a 5,6-dihydro-4H-cyclopenta[b]thiophene-3-carboxylic acid ethyl ester group;
(E) R^{a} represents trifluoromethyl, then R¹ does not represent a 2-methyl-1,2,3,4-tetrahydroisoquinolin-7-yl group;
(F) R^{a} represents iodo, then R¹ does not represent a 2-thiol-4-oxo-4H-quinazolin-3yl group.

2. A compound of formula I, 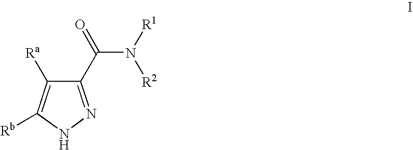
wherein
R¹ represents a bicyclic heterocyclic group, which group is optionally substituted by one or more substituents selected from B¹ and comprises:
(a) 1 to 4 heteroatoms selected from nitrogen, oxygen and sulfur;
(b) a 5- or 6-membered aromatic ring, which ring is attached to the rest of the compound of formula I via the essential —N(R²)— group of the latter; and
(c) a 5- or 6-membered aromatic, or a 4- or 8-membered non-aromatic, ring, which ring is attached to the other ring via two atoms that are common to both rings and are adjacent to each other;
R² represents H or C₁₋₆ alkyl, which latter group is optionally substituted by one or more halo groups;
B¹ represents —OH, cyano, halo, nitro, C₁₋₆ alkyl, (which latter group is optionally substituted by one or more halo atoms), —OR^{3x}, —N(R^{3a})R^{3b}, —C(O)R^{3c}, —C(O)OR^{3d}, —C(O)N(R^{3e})R^{3f}, —N(R^{3g})C(O)R^{3h}, —N(R³ⁱ)C(O)N(R^{3j})R^{3k}, —N(R^{3m})S(O)₂R^{4a}, —S(O)R^{4b}, —OS(O)₂R^{4c}, —S(O)₂N(R³ⁿ)R^{3p} or, provided that it is not attached to a ring that is aromatic in its nature, ═O;
R^{a} and R^{b} independently represent H, halo, cyano, C₁₋₆ alkyl (which alkyl group is optionally substituted by one or more halo or C₁₋₆ alkoxy groups (which alkoxy group may itself be substituted by one or more halo group)), C₁₋₆ alkoxy (which alkoxy group is optionally substituted by one or more halo atoms) or —N(R^{3q})R^{3r};
R^{3a} to R^{3r} and R^{4b} independently represent H or C₁₋₆ alkyl (which alkyl group is optionally substituted by one or more halo atoms);
R^{4a}, R^{4c} and R^{3x} independently represent C₁₋₆ alkyl (which alkyl group is optionally substituted by one or more halo atoms); or
any pair of R^{3a}, R^{3b}, R^{3e} to R^{3r} and R^{4a} may, for example when present on the same or adjacent atoms, be linked together to form, with those, or other relevant, atoms, a 3- or 8-membered ring, optionally containing a further 1 to 3 heteroatoms and/or 1 to 3 unsaturations, which ring is itself optionally substituted by one or more substituents selected from halo and C₁₋₆ alkyl (which alkyl group is optionally substituted by one or more halo atoms); or
when R¹ is substituted by two —OR^{3x} groups that are adjacent to each other, these groups may be linked to form, together with the oxygen atoms to which they are attached, a 5- or 6-membered ring optionally containing 1 further heteroatom and 1 unsaturation, which ring is itself optionally substituted by one or more substituents selected from halo and C₁₋₆ alkyl (which alkyl group is optionally substituted by one or more halo atoms); and
p represents 0, 1 or 2;
or a pharmaceutically-acceptable salt thereof,
provided that, when R^{a} represents trifluoromethyl, R^{b} and R² both represent H, then R¹ does not represent 2-methyl-1,2,3,4-tetrahydroisoquinolin-7-yl group;
for use as a pharmaceutical.

3. A compound as claimed in claim 1 or claim 2, wherein R^{4b} represents C₁₋₆ alkyl (which alkyl group is optionally substituted by one or more halo atoms).

4. A compound as claimed in claim 1, wherein, when R¹ is substituted with two —OR^{3x} groups that are adjacent to each other, then the appropriate pair of R^{3x} groups are not linked as defined in claim 1.

5. A compound as claimed in claim 1, wherein the 5- or 6-membered aromatic ring that is attached to —N(R²)— contains 1 or no heteroatoms.

6. A compound as claimed in claim 1, wherein the 5- or 6-membered aromatic ring that is attached to —N(R²)— is phenyl, pyridyl or thienyl.

7. A compound as claimed in claim 1, wherein the 5- or 5-membered aromatic or 4- to 8-membered non-aromatic ring, which is attached to the other, essential aromatic ring contains less than 3 heteroatoms.

8. A compound as claimed in claim 7, wherein the 5- or 6-membered aromatic or 4- to 8-membered non-aromatic ring is isothiazolyl, cyclopentyl, tetrahydrothiopyranyl, norbornanyl, piperidinyl, tetrahydropyranyl, pyrazinyl, imidazolyl, cycloheptyl, pyridyl, phenyl, pyrrolyl, pyridazinyl, cyclohexyl, thiazolyl, pyrazolyl, dioxolanyl or dioxanyl.

9. A compound as claimed in claim 8, wherein the ring is pyridyl, phenyl, pyrrolyl, pyridazinyl, cyclohexyl, thiazolyl, pyrazolyl, 1,3-dioxolanyl or 1,4-dioxanyl.

10. A compound as claimed in claim 1, wherein R¹ represents benzothiadiazolyl, tetrahydroquinolinyl, dihydrocyclopentapyridyl, tetrahydrothiopyranopyridyl, azatricycloundecatrienyl, tetrahydronaphthyridinyl, dihydropyranopyridyl, quinoxalinyl, benzimidazolyl, tetrahydrocycloheptapyridyl, dihydrothienopyridyl, tetrahydrocycloheptathienyl, naphthyridinyl, quinolinyl, isoquinolinyl, benzodioxolyl, indolyl, cinnolinyl, benzodioxanyl, tetrahydrobenzothienyl, benzothiazolyl or indazolyl.

11. A compound as claimed in claim 10, wherein R¹ represents quinolinyl, isoquinolinyl, benzodioxolyl, indolyl, cinnolinyl, benzodioxanyl, tetrahydrobenzothienyl, benzothiazolyl or indazolyl.

12. A compound as claimed in claim 1, wherein the optional substituent(s) on the R¹ group is/are selected from C₁₋₃ alkoxy, —O, —OH, —SH, —C(O)R^{3c}, in which R^{3c} represents C₁₋₃ alkyl, two adjacent —OR^{3x} groups that are linked together to form a 5- or 6-membered ring, halo, C₁₋₄ alkyl (optionally substituted by one or more halo atoms), C(O)OR^{3d}, in which R^{3d} represents C₁₋₄ alkyl, and cyano.

13. A compound as claimed in claim 12, wherein the optional substituent(s) is/are selected from halo, C₁₋₃ alkyl (optionally substituted by one or more halo atoms), C(O)OR^{3d}, in which R^{3d} represents C₁₋₃ alkyl, and cyano.

14. A compound as claimed in claim 1, wherein R² represents H.

15. A compound as claimed in claim 1, wherein R^{a} and R^{b} independently represent halo, H or C₁₋₃ alkyl.

16. A compound as claimed in claim 15, wherein R^{a} and R^{b} independently represent H or C₁₋₃ alkyl.

17. A pharmaceutical formulation including a compound of formula I, as defined in claim 1 or claim 2, or a pharmaceutically-acceptable salt thereof, in admixture with a pharmaceutically acceptable adjuvant, diluent or carrier.

18. The method which comprises administering a compound of formula I, as defined in claim 1, but without the provisos, or a pharmaceutically-acceptable salt thereof, for the treatment of a disease in which inhibition of the activity of a lipoxygenase is desired and/or required.

19. The method as claimed in claim 18, wherein the lipoxygenase is 15-lipoxygenase.

20. The method as claimed in claim 19, wherein the disease is inflammation and/or has an inflammatory component.

21. The method as claimed in claim 20 wherein the inflammatory disease is asthma, chronic obstructive pulmonary disease, pulmonary fibrosis, an allergic disorder, rhinitis, inflammatory bowel disease, an ulcer, inflammatory pain, fever, atherosclerosis, coronary artery disease, vasculitis, pancreatitis, arthritis, osteroarthritis, rheumatoid arthritis, conjunctivitis iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

22. A method of treatment of a disease in which inhibition of the activity of a lipoxygenase is desired and/or required, which method comprises administration of a therapeutically effective amount of a compound of formula I as defined in claim 1, but without the provisos, or a pharmaceutically-acceptable salt thereof, to a patient suffering from, or susceptible to, such a condition.

23. A combination product comprising:
(A) a compound of formula I as defined in claim 1, but without the provisos, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

24. A combination product as claimed in claim 23 which comprises a pharmaceutical formulation including a compound of formula I as defined in claim 1, but without the provisos, or a pharmaceutically-acceptable salt thereof, another therapeutic agent that is useful in the treatment of inflammation and a pharmaceutically-acceptable adjuvant, diluent or carrier.

25. A combination product as claimed in claim 23 which comprises a kit of parts comprising components:
(a) a pharmaceutical formulation including a compound of formula I as defined in claim 1, but without the provisos, or a pharmaceutically-acceptable salt thereof; in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

26. A process for the preparation of a compound of formula I as defined in claim 1, which comprises:
(i) for compounds of formula I in which R^{b} represents C₁₋₆ alkyl, optionally substituted as defined in claim 1, to halo, reaction of a corresponding compound of formula I in which R^{b} represents hydrogen, with an appropriate base, followed by quenching with:
(a) for compounds of formula I in which R^{b} represents an optionally substituted C₁₋₆ alkyl group, a compound of formula II,
R^{c}L^{1a} II
wherein R^{c} represents C₁₋₆ alkyl (which alkyl group is optionally substituted by one or more halo or C₁₋₆ alkoxy groups (which alkoxy group may itself be substituted by one or more halo group)), and L^{1a} represents a suitable leaving group; or
(b) for compounds of formula I in which R^{b} represents halo, a compound that provides a source of halide ions;
(ii) reaction of a compound of formula III, 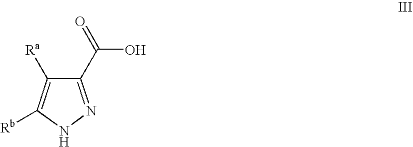
or a N-protected and/or O-protected derivative thereof, wherein R^{a} and R^{b} are as defined in claim 1, with a compound of formula IV,
HN(R¹)(R²) IV
wherein R¹ and R² are as defined in claim 1;
(ii) reaction of a compound of formula V, 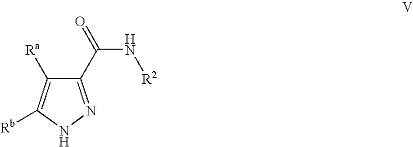
or a N-protected derivative thereof, wherein R^{a}, R^{b} and R² are as defined in claim 1, with a compound of formula VI,
R¹-L² VI
wherein L² represents a suitable leaving group and R¹ is as defined in claim 1;
(iv) for compounds of formula I in which R^{b} represents hydrogen and R^{a} is as defined in claim 1, reaction of a compound of formula VII, 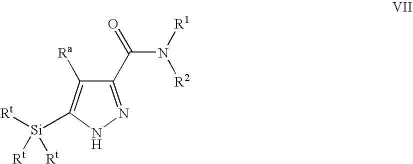
wherein each R¹ independently represents a C₁₋₆ alkyl group or an aryl group, and R^{a}, R¹ and R² are as defined in claim 1, with an appropriate reagent for the removal of the silyl group;
(v) reaction of a compound of formula VIII, 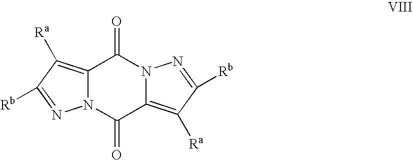
wherein R^{a} and R^{b} are as defined in claim 1, with a compound of formula IV as defined above;
(vi) for compounds of formula I in which one of R^{a} and R^{b} represents an optionally substituted C₁₋₆ alkyl group and the other represents H, reaction of a corresponding compound of formula I in which one of R^{a} and R^{b} represents bromo or iodo and the other represents H (as appropriate) with a suitable organolithium base, followed by quenching with a compound of formula IV as defined above;
(vii) for compounds of formula I in which R^{a} and R^{b} represent C₁₋₆ alkoxy (optionally substituted as described in claim 1), reaction of a compound corresponding to a compound of formula I but in which in place of the relevant substituents, R^{a} and/or R^{b} (as appropriate), (a) hydroxy group(s) is/are present, with a compound of formula II as defined above, in which R^{c} represents C₁₋₆ alkyl (optionally substituted by one or more halo substituents), or (for the introduction of a methoxy group at R^{a} and R^{b}) with diazomethane,
(viii) for compounds of formula I in which R^{a} and/or R^{b} represents —N(R^{3q})R^{3r} in which one of R^{3q} or R^{3r} represents H and the other represents methyl or C₂₋₆ alkyl (which latter group is optionally substituted by one or more halo atoms), reaction of a corresponding compound of formula I in which R^{a} and/or R^{b} represents —NH₂, with a compound of formula IX,
R^{3s}C(O)H IX
wherein R^{3s} represents H or C₁₋₅ alkyl (which alkyl group is optionally substituted by one or more halo atoms);
(ix) for compounds of formula I in which R^{a} and/or R^{b} represent —N(R^{3q})^{3r} in which at least one of R^{3q} and R^{3r} represents C₁₋₆ alkyl (optionally substituted as defined in claim 1) and the other represents H or C₁₋₆ alkyl (optionally substituted as defined in claim 1), reaction of a corresponding compound of formula I in which R^{a} and/or R^{b} represent(s) —NH₂ (as appropriate) with a compound of formula II in which R^{c} represents C₁₋₆ alkyl optionally substituted by one or more halo substituents;
(x) for compounds of formula I in which R^{a} and/or R^{b} represent —N(R^{3q})R^{3r} in which R^{3q} and R^{3r} are linked together to form a 3- to 8-membered ring, reaction of a corresponding compound of formula I in which R^{a} and/or R^{b} represents —NH₂ (as appropriate) with a compound of formula IXA,
L^{1a}-Z-L^{1a} IXA
wherein Z represents a C₂₋₇ alkylene chain (which alkylene chain optionally contains 1 to 3 heteroatoms and/or 1 to 3 unsaturations, and is optionally substituted by one or more groups selected from halo and C₁₋₆ alkyl (which alkyl group is optionally substituted by one or more halo atoms)), and L^{1a} is as defined above;
(xi) for compounds of formula I in which R^{a} and/or R^{b} represents cyano or 1-alkynyl, reaction of a corresponding compound of formula I in which R^{a} and/or R^{b} (as appropriate) represents halo with a compound which is a source of cyano anions for the introduction of the cyano group, or with a 1-alkyne for the introduction of the 1-alkynyl group; or
(xii) for compounds of formula I in which R² represents H, reaction of a compound of formula IXB, 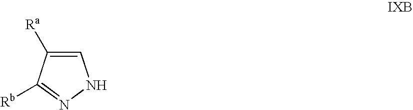
or a N-protected derivative thereof, wherein R^{a} and R^{b} are as defined in claim 1, with a suitable base, followed by reaction with a compound of formula IXC,
R¹—N═C═O IXC
wherein R¹ is as defined in claim 1, followed by quenching with a suitable proton source.
